(19) United States
(12) Patent Application Publication
(10) Pub. No.: US 2021/0052741 A1
(43) Pub. Date: Feb. 25, 2021

(54) GENE THERAPY METHODS AND COMPOSITIONS USING AUXOTROPHIC REGULATABLE CELLS

(71) Applicants: AUXOLYTIC LTD, London (GB); The Board of Trustees of the Leland Stanford Junior University, Stanford, CA (US)

(72) Inventors: James PATTERSON, London (GB); Matthew PORTEUS, Stanford, CA (US); Volker WIEBKING, Menlo Park, CA (US)

(73) Assignees: AUXOLYTIC LTD, London (GB); The Board of Trustees of the Leland Stanford Junior University, Stanford, CA (US)

(21) Appl. No.: 17/088,932

(22) Filed: Nov. 4, 2020

Related U.S. Application Data
(63) Continuation of application No. PCT/US2019/031699, filed on May 10, 2019.
(60) Provisional application No. 62/669,848, filed on May 10, 2018.

(51) Int. Cl. A61K 48/00; A61K 35/17; C12N 15/90; C12N 5/0783; A61P 3/00

(52) U.S. Cl. CPC A61K 48/005; A61K 35/17; C12N 15/907; C12N 2800/80; A61P 3/00; C12N 2310/20; C12N 2510/00; C12N 5/0636

(57) ABSTRACT

The present disclosure provides compositions and methods for producing and using modified auxotrophic host cells for improved gene therapy involving administration of an auxotrophic factor.

### CROSS-REFERENCE TO RELATED APPLICATIONS

The present application is a continuation of International Application No. PCT/US2019/031699, filed May 10, 2019, which claims priority to U.S. Provisional Application No. 62/669,848, filed May 10, 2018, the contents of which are hereby incorporated by reference in their entirety.

### REFERENCE TO A “SEQUENCE LISTING,” A TABLE, OR A COMPUTER PROGRAM LISTING APPENDIX SUBMITTED ON A COMPACT DISK

The present application is being filed along with a Sequence Listing in electronic format. The Sequence Listing file, entitled 079445-1213703-002910US_SL.txt, was created on Oct. 27, 2020, and is 2,019 bytes in size. The information in electronic format of the Sequence Listing is incorporated herein by reference in its entirety.

### FIELD OF THE DISCLOSURE

The disclosure herein relates to gene therapy methods, compositions and kits with improved efficacy and safety.

### BACKGROUND

Cell therapies have been shown to provide promising treatments. Yet, reintroduction of modified cells into a human host carries risks including immune reactions, malignant transformation, or overproduction or lack of control of transgenes.

Several approaches of genetic engineering enable the control over functions of human cells like cell signaling, proliferation or apoptosis (See, Bonifant, et al. Mol. Ther.—Oncolytics 3, 16011 (2016); Sockolosky et al., (2018). Science (80-.). 359, 1037-1042 Tey, (2014) Clin. Transl. Immunol. 3, e17; each of which is hereby incorporated by reference in its entirety) and made it possible to control even severe side effects of cell therapies (Bonifant et al., 2016). Despite these advances, other applications have been prevented from gaining widespread application, e.g. the use of engineered pluripotent cells for regenerative medicine (See, Ben-David and Benvenisty, 2011, Nat. Rev. Cancer 11, 268-277.; Lee et al., 2013, Nat. Med. 19, 998-1004; Porteus, M. (2011) Mol. Ther. 19, 439-441; each of which is hereby incorporated by reference in its entirety), due to the fact that control systems that rely on the introduction of a genetically encoded control mechanism into the cell have multiple limitations (Tey, 2014).

Two of the major problems that can arise are “leakiness”, i.e. low-level activity of the mechanism in the absence of its trigger (see, Ando et al. (2015) Stem Cell Reports 5, 597-608, which is hereby incorporated by reference in its entirety), and the lack of removal of the entire cell population upon activation of the mechanism (see, Garin et al. (2001) Blood 97, 122-129; Di Stasi et al. (2011) N Engl J Med 365, 1673-1683; Wu et al. (2014) N Engl J Med 365, 1673-1683; agyu et al. (2015) Mol. Ther. 23, 1475-1485; each of which is hereby incorporated by reference in its entirety), due to several escape mechanisms from external control. For example, the transgene that is introduced by viral transduction can be silenced from expression by the cell (see, Sulkowski et al. (2018) Switch. Int. J. Mol. Sci. 19, 197, which is hereby incorporated by reference in its entirety) or the cell can develop resistance towards the effector mechanism (See, Yagyu et al. (2015) Mol. Ther. 23, 1475-1485, which are hereby incorporated by reference in its entirety). Another concern is the mutation of the transgene in cell types with genetic instability, e.g. cell lines that are cultured for prolonged periods of time or tumor cell lines (Merkle et al. (2017) Nature 545, 229-233; D'Antonio et al. (2018) Cell Rep. 24, 883-894; each of which is hereby incorporated by reference in its entirety). Moreover, primary cell populations often retain their functionality for only limited time in ex vivo culture and many types cannot be purified by clonal isolation.

Existing modes of safety switches also have a number of risks, such as (1) transgene insertion into a tumor suppressor leading to oncogenic transformation of the cell line, and (2) transgene insertion into an epigenetically silenced region leading to lack of expression and thus efficacy, or subsequent epigenetic silencing of the transgene after insertion. Genome instability is a common phenotype in oncogenic transformation of a cell. Further, a point mutation or genetic loss of an exogenous suicide switch would be quickly selected for and amplified. A safety switch based on targeting a signaling pathway of the cell depends on the physiology of the cell. For example, a cell that is in “pro-survival” mode may express caspase inhibitors, preventing cell death upon suicide switch induction.

An especially attractive application of gene therapy involves the treatment of disorders that are either caused by an insufficiency of a gene product or that are treatable by increased expression of a gene product, for example a therapeutic protein, antibody or RNA.

Recent advances allow the precise modification of the genome of human cells. This genetic engineering enables a wide range of applications, but also requires new methods to control cell behavior. An alternative control system for cells is auxotrophy that can be engineered by targeting a gene in metabolism. This concept has been explored for microorganisms (see, Steidler et al. (2003) Nat. Biotechnol. 21, 785-789, which is hereby incorporated by reference in its entirety) and has been broadly used as a near universal research tool by yeast geneticists. It would be particularly powerful in mammalian cells if it is created by knockout of a gene instead of by introduction of a complex control mechanism, and if the auxotrophy is towards a non-toxic compound that is part of the cell's endogenous metabolism. This could be achieved by disruption of an essential gene in a metabolic pathway, allowing the cell to function only if the product of that pathway is externally supplied and taken up by the cell from its environment. Furthermore, if the respective gene is also involved in the activation of a cytotoxic agent, the gene knockout (KO) would render the cells resistant to that drug, thereby enabling the depletion of non-modified cells and purification of the engineered cells in a cell population. Several monogenic inborn errors of metabolism can be treated by supply of a metabolite and can therefore be seen as models of human auxotrophy.

### SUMMARY OF THE DISCLOSURE

Disclosed herein, in some embodiments, are donor templates comprising (a) one or more nucleotide sequences homologous to a fragment of an auxotrophy-inducing locus, or homologous to the complement of said auxotrophy-inducing locus, and (b) a transgene encoding a therapeutic factor, optionally linked to an expression control sequence. In some instances, the donor template is single stranded. In some instances, the donor template is double stranded. In some instances, the donor template is a plasmid or DNA fragment or vector. In some instances, the donor template is a plasmid comprising elements necessary for replication, optionally comprising a promoter and a 3′ UTR. Disclosed herein, in some embodiments, are vectors comprising (a) one or more nucleotide sequences homologous to a fragment of the auxotrophy-inducing locus, or homologous to the complement of said auxotrophy-inducing locus, and (b) a transgene encoding a therapeutic factor. In some instances, the vector is a viral vector. In some instances, the vector is selected from the group consisting of retroviral, lentiviral, adenoviral, adeno-associated viral and herpes simplex viral vectors. In some instances, the vector further comprises genes necessary for replication of the viral vector. In some instances, the transgene flanked on both sides by nucleotide sequences homologous to a fragment of the auxotrophy-inducing locus or the complement thereof. In some instances, the auxotrophy-inducing locus is a gene encoding a protein that is involved in synthesis, recycling or salvage of an auxotrophic factor. In some instances, the auxotrophy-inducing locus is within a gene in Table 1 or within a region that controls expression of a gene in Table 1. In some instances, the auxotrophy-inducing locus is within a gene encoding uridine monophosphate synthetase. In some instances, the auxotrophy-inducing locus is within a gene encoding holocarboxylase synthetase. In some instances, the nucleotide sequence homologous to a fragment of the auxotrophy-inducing locus is 98% identical to at least 200 consecutive nucleotides of the auxotrophy-inducing locus. In some instances, the nucleotide sequence homologous to a fragment of the auxotrophy-inducing locus is 98% identical to at least 200 consecutive nucleotides of human uridine monophosphate synthetase or holocarboxylase synthetase or any of the genes in Table 1. In some instances, the donor template or vector further comprises an expression control sequence operably linked to said transgene. In some instances, the expression control sequence is a tissue-specific expression control sequence. In some instances, the expression control sequence is a promoter or enhancer. In some instances, the expression control sequence is an inducible promoter. In some instances, the expression control sequence is a constitutive promoter. In some instances, the expression control sequence is a posttranscriptional regulatory sequence. In some instances, the expression control sequence is a microRNA. In some instances, the donor template or vector further comprises a marker gene. In some instances, the marker gene comprises at least a fragment of NGFR or EGFR, at least a fragment of CD20 or CD19, Myc, HA, FLAG, GFP, an antibiotic resistance gene. In some instances, the transgene is selected from the group consisting of hormones, cytokines, chemokines, interferons, interleukins, interleukin-binding proteins, enzymes, antibodies, Fc fusion proteins, growth factors, transcription factors, blood factors, vaccines, structural proteins, ligand proteins, receptors, cell surface antigens, receptor antagonists, and co-stimulating factors, structural proteins, cell surface antigens, ion channels an epigenetic modifier or an RNA editing protein. In some instances, the transgene encodes a T cell antigen receptor. In some instances, the transgene encodes an RNA, optionally a regulatory microRNA.

Disclosed herein, in some embodiments, are nuclease systems for targeting integration of a transgene to an auxotrophy-inducing locus comprising a cas9 protein, and a guide RNA specific for an auxotrophy-inducing locus. Disclosed herein, in some embodiments, are nuclease system for targeting integration of a transgene to an auxotrophy-inducing locus comprising a meganuclease specific for said auxotrophy-inducing locus. In some instances, the meganuclease is a ZFN or TALEN. In some instances, the nuclease system further comprises a donor template or vector disclosed herein.

Disclosed herein, in some embodiments, are modified host cell ex vivo, comprising a transgene encoding a therapeutic factor integrated at an auxotrophy-inducing locus, wherein said modified host cell is auxotrophic for an auxotrophic factor and capable of expressing the therapeutic factor. In some instances, the modified host cell is a mammalian cell. In some instances, the modified host cell is a human cell. In some instances, the modified host cell is an embryonic stem cell, a stem cell, a progenitor cell, a pluripotent stem cell, an induced pluripotent stem (iPS) cell, a somatic stem cell, a differentiated cell, a mesenchymal stem cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell or a peripheral blood mononuclear cell (PBMC). In some instances, the modified host cell is derived from cells from a subject to be treated with the modified host cells.

Disclosed herein, in some embodiments, are methods of producing a modified mammalian host cell comprising (a) introducing into said mammalian host cell one or more nuclease systems that targets and cleaves DNA at the auxotrophy-inducing locus, or a nucleic acid encoding one or more components of said one or more nuclease systems, and (b) a donor template or vector disclosed herein. In some instances, the methods further comprising introducing a second nuclease or second guide RNA to target and cleave DNA at a second genomic locus, or a nucleic acid encoding said second nuclease or second guide RNA, and optionally (b) a second donor template or vector.

Disclosed herein, in some embodiments, are methods of targeting integration of a transgene to an auxotrophy-inducing locus in a mammalian cell ex vivo comprising contacting said mammalian cell with a donor template or vector disclosed herein, and a nuclease. In some instances, the nuclease is a ZFN. In some instances, the nuclease is a TALEN.

Disclosed herein, in some embodiments, are methods of producing a modified mammalian host cell comprising introducing into said mammalian host cell with: (a) a Cas9 polypeptide, or a nucleic acid encoding said Cas9 polypeptide, (b) a guide RNA specific to an auxotrophy-inducing locus, or a nucleic acid encoding said guide RNA, and (c) a donor template or vector disclosed herein. The methods further comprising introducing into said mammalian host cell with (a) a second guide RNA specific to a second auxotrophy-inducing locus, or a nucleic acid encoding said guide RNA, and optionally (b) a second donor template or vector.

Disclosed herein, in some embodiments, are methods of targeting integration of a transgene to an auxotrophy-inducing locus in a mammalian cell ex vivo comprising contacting said mammalian cell with a donor template or vector disclosed herein, a cas9 polypeptide, and a guide RNA. In some instances, the guide RNA is a chimeric RNA. In some instances, the guide RNA comprises two hybridized RNAs. In some instances, the methods produce one or more single stranded breaks within the auxotrophy-inducing locus. In some instances, the methods produce a double stranded break within the auxotrophy-inducing locus. In some instances, the auxotrophy-inducing locus is modified by homologous recombination using said donor template or vector. In some instances, the steps (a) and (b) are carried out before or after expanding said cells, and optionally culturing said cells. In some instances, the methods further comprising (c) selecting cells that contain the transgene integrated into the auxotrophy-inducing locus. In some instances, the selecting comprises (i) selecting cells that require the auxotrophic factor to survive and optionally (ii) selecting cells that comprise the transgene integrated into the auxotrophy-inducing locus. In some instances, the auxotrophy-inducing locus is a gene encoding uridine monophosphate synthetase and the cells are selected by contacting with 5-FOA.

Disclosed herein, in some embodiments, are sterile composition containing said donor template or vector, or said nuclease system, and sterile water or a pharmaceutically acceptable excipient. Disclosed herein, in some embodiments, are sterile composition comprising the modified mammalian host cell and sterile water or a pharmaceutically acceptable excipient. Disclosed herein, in some embodiments, are kit containing said donor template or vector or nuclease system or modified host cell, or a combination thereof, of any of the preceding claims, optionally with a container or vial.

Disclosed herein, in some embodiments, are methods of expressing a therapeutic factor in a subject comprising (a) administering the modified host cells, (b) optionally administering a conditioning regime to permit modified cells to engraft, and (c) administering the auxotrophic factor. In some instances, the modified host cells and auxotrophic factor are administered concurrently. In some instances, the modified host cells and auxotrophic factor are administered sequentially. In some instances, administration of said auxotrophic factor is continued regularly for a period of time sufficient to promote expression of the therapeutic factor. In some instances, administration of said auxotrophic factor is decreased to decrease expression of the therapeutic factor. In some instances, administration of said auxotrophic factor is increased to increase expression of the therapeutic factor. In some instances, administration of said auxotrophic factor is discontinued to create conditions that result in growth inhibition or death of the modified host cells. In some instances, administration of said auxotrophic factor is temporarily interrupted to create conditions that result in growth inhibition of the modified host cells. In some instances, administration of said auxotrophic factor is continued for a period of time sufficient to exert a therapeutic effect in a subject. In some instances, the modified host cell is regenerative. In some instances, the administration of the modified host cell comprises localized delivery. In some instances, the administration of the auxotrophic factor comprises systemic delivery. In some instances, the host cell prior to modification is derived from the subject to be treated.

Disclosed herein, in some embodiments, are methods of treating a subject with a disease, a disorder, or a condition comprising administering to the subject (a) said modified host cells and (b) said auxotrophic factor in an amount sufficient to produce expression of a therapeutic amount of the therapeutic factor. In some instances, the disease, the disorder, or the condition is selected from the group consisting of cancer, Parkinson's disease, graft versus host disease (GvHD), autoimmune conditions, hyperproliferative disorder or condition, malignant transformation, liver conditions, genetic conditions including inherited genetic defects, juvenile onset diabetes mellitus and ocular compartment conditions. In some instances, the disease, the disorder, or the condition affects at least one system of the body selected from the group consisting of muscular, skeletal, circulatory, nervous, lymphatic, respiratory endocrine, digestive, excretory, and reproductive systems.

Disclosed herein, in some embodiments, are uses of a modified host cell disclosed herein for treatment of a disease, disorder or condition. Disclosed herein, in some embodiments, are the modified host cell disclosed herein for use in administration to humans, or for use in treating a disease, a disorder or a condition.

Disclosed herein, in some embodiments, are auxotrophic factor for use in administration to a human that has received a modified human host cell.

Disclosed herein, in some embodiments, are methods of alleviating or treating a disease or disorder in an subject in need thereof, the method comprising administering to the subject: (a) a composition comprising modified host cell comprising a transgene encoding a protein integrated at an auxotrophy-inducing locus, wherein the modified host cell is auxotrophic for an auxotrophic factor; and (b) the auxotrophic factor in an amount sufficient to produce therapeutic expression of the protein. In some instances, the auxotrophy-inducing locus is within a gene encoding uridine monophosphate synthetase (UMPS). In some instances, the auxotrophic factor is uridine. In some instances, the auxotrophy-inducing locus is within a gene encoding holocarboxylase synthetase (HLCS). In some instances, the auxotrophic factor is biotin. In some instances, the protein is an enzyme. In some instances, the protein is an antibody. In some instances, the modified host cell is an embryonic stem cell, a stem cell, a progenitor cell, a pluripotent stem cell, an induced pluripotent stem (iPS) cell, a somatic stem cell, a differentiated cell, a mesenchymal stem cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell or a peripheral blood mononuclear cell (PBMC). In some instances, the modified host cell is a mammalian cell. In some instances, the mammalian cell is a human cell. In some instances, the modified host cell is derived from the subject to be treated with the modified host cell. In some instances, the composition and the auxotrophic factor are administered concurrently. In some instances, the composition and the auxotrophic factor are administered sequentially. In some instances, the composition is administered before the auxotrophic factor. In some instances, the composition and the auxotrophic factor are administered concurrently. In some instances, administration of the auxotrophic factor is continued regularly for a period of time sufficient to promote therapeutic expression of the protein. In some instances, administration of the auxotrophic factor is decreased to decrease expression of the protein. In some instances, administration of the auxotrophic factor is increased to increase expression of the protein. In some instances, discontinued administration of the auxotrophic factor induces growth inhibition or cell death of the modified host cell. In some instances, administration of the auxotrophic factor is continued for a period of time sufficient to exert a therapeutic effect in the subject. In some instances, the modified host cell is regenerative. In some instances, the administration of the composition comprises localized delivery. In some instances, the administration of the auxotrophic factor comprises systemic delivery. In some instances, the disease is a lysosomal storage disease (LSD). In some instances, the LSD is Gaucher's Disease (Type 1/2/3), MPS2 (Hunter's) disease, Pompe disease, Fabry disease, Krabbe disease, Hypophosphatasia, Niemann-Pick disease type A/B, MPS1, MPS3A, MPS3B, MPS3C, MPS3, MPS4, MPS6, MPS7, Phenylketonuria, MLD, Sandhoff disease, Tay-Sachs disease, or Battens disease. In some instances, the enzyme is Glucocerebrosidase, Idursulfase, Alglucosidase alfa, Agalsidase alfa/beta, Galactosylceramidase, Asfotase alfa, Acid Sphingomyelinase, Laronidase, heparan N-sulfatase, alpha-N-acetylglucosaminidase, heparan-α-glucosaminide N-acetyltransferase, N-acetylglucosamine 6-sulfatase, Elosulfase alfa, Glasulfate, B-Glucoronidase, Phenylalanine hydroxylase, Arylsulphatase A, Hexosaminidase-B, Hexosaminidase-A, or tripeptidyl peptidase 1. In some instances, the disease is Friedreich's ataxia, Hereditary angioedema, or Spinal muscular atrophy. In some instances, the protein is frataxin, C1 esterase inhibitor (which may also be referred to as HAEGAARDA® subcutaneous injection) or SMN1.

Various embodiments described herein provide a method of reducing the size of a tumor or reducing a rate of growth of a tumor in a subject, the method comprising: administering to the subject a modified human host cell as described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the subject matter encompassed by the present disclosure are set forth with particularity in the appended claims. A better understanding of the features and advantages of the present disclosure will be obtained by reference to the following detailed description that sets forth illustrative embodiments, in which the principles of the subject matter encompassed by the disclosure herein are utilized, and the accompanying drawings of which:

FIG. 1A and FIG. 1B exemplify the effect of serum on optimal recovery post-electroporation. FIG. 1A is an exemplary schematic of assay used to determine optimal electroporation recovery conditions. Following electroporation, cells were supplied with/without serum, 5-fluoroorotic acid (5-FOA), or an exogenous uracil source (uridine). FIG. 1B illustrates cell counts by CytoFLEX flow cytometer (Beckman Coulter) after 4 days of recovery post electroporation in indicated media conditions. The figure shows cells administered serum, mock edited cells treated with/without 5-FOA with no serum, and uridine monophosphate synthetase (UMPS) knockout cells treated with/without 5-FOA without serum.

FIG. 2A-FIG. 2F exemplifies that maintenance and growth of UMPS InDel containing cells requires an exogenous uracil source. FIG. 2A is an exemplary schematic of the procedure used to assay for growth of UMPS or mock edited T cells following electroporation and recovery. FIG. 2B illustrates tracking of indels by decomposition (TIDE) analysis of UMPS InDels in indicated culture conditions. TIDE analysis was performed on sanger sequencing of UMPS locus with oligonucleotides UMPS-O-1 and UMPS-O-2. FIG. 2C illustrates percentage of alleles containing frameshift InDels analyzed by TIDE performed on day 8. FIG. 2D illustrates predicted absolute numbers of cells at day 8 containing alleles identified by TIDE. FIG. 2E illustrates time course of cell counts with/without UMP. FIG. 2F illustrates time course of cell counts with/without uridine.

FIG. 3A-FIG. 3C exemplifies that 5-FOA is less toxic in UMPS targeted cell lines. FIG. 3A is an exemplary schematic of 5-FOA selection procedure. FIG. 3B and FIG. 3C illustrate cell counts after 4 days of 5-FOA selection in indicated culture conditions. In FIG. 3B and FIG. 3C, the mock results are represented by the left bar for each culture condition, and the results for UMPS-7 are shown by the right bar for each culture condition.

FIG. 4A-FIG. 4D exemplifies that 5-FOA selected, UMPS targeted cell lines exhibit optimum growth only in the presence of an exogenous uracil source. FIG. 4A is an exemplary schematic of protocol for the demonstration of uracil auxotrophy. Cell cultures were split following 4-day selection in 5-FOA into test media and grown for 4 further days before cell counting. FIG. 4B-FIG. 4D illustrate cell counts of 5-FOA selected cells in exogenous uracil (UMP or uridine) containing or deficient media.

FIG. 5A exemplifies InDel quantification performed at the UMPS locus by the ICE analysis. FIG. 5B exemplifies proliferation of T cells after mock treatment, CCR5 knockout or UMPS knockout. FIG. 5C illustrates proliferation of T cells with UMPS knockout with or without UMP or Uridine. FIG. 5D illustrates InDel frequency on day 8 after UMPS knockout with different culture conditions. FIG. 5E illustrates the frequency of InDels that are predicted to lead to a frameshift.

FIG. 6A exemplifies DNA donor constructs for targeting of the UMPS locus. FIG. 6B illustrates expression of surface markers after targeting of K562 cells. FIG. 6C exemplifies targeting approach to integrate Nanoluciferase and green fluorescent protein (GFP) into the HBB locus. FIG. 6D illustrates expression of the 3 integrated markers in K562 cells before cell sorting.

FIG. 6E illustrates K562 cell growth and cell counts on day 8 when cultured in the presence of different Uridine concentrations. FIG. 6F illustrates selection of UMPS^{KO/KO} cells during culture with 5-FOA. FIG. 6G illustrates proliferation of UMPS^{KO/KO} cells in the presence of 5-FOA.

FIG. 7A exemplifies surface marker expression after UMPS targeting of T cells. FIG. 7B illustrates auxotrophic growth of UMPS^{KO} or wild-type (WT) T cells. FIG. 7C illustrates that 5-FOA selects for T cells with UMPS knockout.

Groups were compared by statistical tests as indicated using Prism 7 (GraphPad). Asterisks indicate levels of statistical significance: *=p<0.05, **=p<0.01, ***=p<0.001, and ****=p<0.0001.

### DETAILED DESCRIPTION

### I. Introduction

Recent advances allow the precise modification of the genome of human cells. This genetic engineering enables a wide range of applications, but also requires new methods to control cell behavior. An alternative control system for cells is auxotrophy that can be engineered by targeting a gene in metabolism. The approach described herein of genetically engineering auxotrophy by disruption of a central gene of metabolism is an alternative paradigm to create an external control mechanism over cell function which has not been explored for human cells. By disrupting a key gene in pyrimidine metabolism, a passive containment system was created (Steidler et al., 2003), which is an addition and alternative to the already existing toolbox of systems for human cells that circumvents their previously mentioned limitations. It enables the control over growth of human cells through the addition or withdrawal of the non-toxic substance uridine. Auxotrophy has previously been engineered in microorganisms, e.g. towards an unnatural substance by introduction of an engineered gene circuit (see, Kato, Y. (2015) An engineered bacterium auxotrophic for an unnatural amino acid: a novel biological containment system. PeerJ 3, e1247, which is hereby incorporated by reference in its entirety) or towards pyrimidines by knockout of a bacterial gene (see, Steidler et al. (2003) Nat. Biotechnol. 21, 785-789, which is hereby incorporated by reference in its entirety). The latter concept is appealing, since it relies on the knockout of a gene instead of the introduction of complex expression cassettes, which impedes the cell from reversing the genetic modification or the development of resistance mechanisms, and therefore addresses this challenge of alternative systems. The fact that pyrimidine nucleosides and nucleotides play an essential role in a wide array of cellular processes, including DNA and RNA synthesis, energy transfer, signal transduction and protein modification (see, van Kuilenburg, A. B. P. and Meinsma, R. (2016). Biochem. Biophys. Acta-Mol. Basis Dis. 1862, 1504-1512, which is hereby incorporated by reference in its entirety) makes their synthesis pathway a theoretically appealing target.

Human cells are naturally auxotrophic for certain compounds like amino acids that they have to acquire, either from external sources or symbiotic organisms (See, Murray, P. J. (2016). Nat. Immunol. 17, 132-139, which is hereby incorporated by reference in its entirety). Additionally, auxotrophy is a natural mechanism to modulate the function of immune cells, e.g. by differential supply or depletion of the metabolite that the cells are auxotrophic for (See, Grohmann et al., (2017). Cytokine Growth Factor Rev. 35, 37-45, which is hereby incorporated by reference in its entirety). Cellular auxotrophy also plays an important role in mechanisms of defense against malignant growth, e.g. in the case of macrophages that inhibit tumor growth by scavenging arginine (Murray, 2016). In addition, several malignant cell types have been shown to be auxotrophic for certain metabolites (see, Fung, M. K. L. and Chan, G. C. F. (2017). J. Hematol. Oncol. 10, 144, which is hereby incorporated by reference in its entirety), which is exploited by the therapeutic depletion of asparagine for the treatment of leukemia patients (See, Hill et al., (1967). JAMA 202, 882).

In addition to the previously developed containment strategies for microorganisms, the approach described herein using gene editing based on Cas9 ribonucleoprotein (RNP)/rAAV6 allows for highly efficient engineering of a primary and therapeutically relevant human cell type. Auxotrophy and resistance to 5-FOA are inherent to all cells with complete disruption of the UMPS gene, but to show proof-of-concept, the identification of the populations was facilitated with bi-allelic knockout by targeted integration of selection markers. The recent development of methods that allow the efficient targeted modification of primary human cells (Bak et al. (2017).

Multiplexed genetic engineering of human hematopoietic stem and progenitor cells using CRISPR/Cas9 and AAV6. Elife 6, e27873; Bak, et al. (2018). Nat. Protoc. 13, 358-376; Porteus, M. H. and Baltimore, D. (2003). Science (80-.). 300, 763-763; each of which is hereby incorporated by reference in its entirety) together with the establishment of metabolic auxotrophy lays the foundation for further development of therapeutic approaches in settings where the use of human cells is necessary, e.g. in the use of stem cells or stem-cell derived tissues or other autologous somatic cells with specific effector functions and reduced immunogenicity. Notably, constructs and reagents have been used that would facilitate expedited clinical translation, e.g. selection markers tNGFR and tEGFR in the targeting constructs, which avoid immunogenicity, and uridine supplied in the in vivo model using its FDA-approved prodrug.

Engineered mechanisms to control cell function have the additional challenge of selecting an entirely pure population of cells that express the proteins mediating the control mechanism. The possibility of selecting the engineered cells by rendering them resistant to a cytotoxic agent is particularly appealing since it can substantially increase efficiency by allowing the creation of a highly pure population of cells that can be controlled using a non-toxic substance, and the removal of a gene crucial for the function of a vital metabolic pathway prevents cells from developing escape mechanisms. Therefore, this method offers several advantages over existing control mechanisms in settings where genetic instability and the risk of malignant transformation play a role and where even small numbers of cells that escape their containment can have disastrous effects, e.g. in the use of somatic or pluripotent stem cells.

This concept has been explored for microorganisms (Steidler et al., 2003) and has been broadly used as a near universal research tool by yeast geneticists. It would be particularly powerful in mammalian cells if it is created by knockout of a gene instead of by introduction of a complex control mechanism, and if the auxotrophy is towards a non-toxic compound that is part of the cell's endogenous metabolism. This could be achieved by disruption of an essential gene in a metabolic pathway, allowing the cell to function only if the product of that pathway is externally supplied and taken up by the cell from its environment. Furthermore, if the respective gene is also involved in the activation of a cytotoxic agent, the gene knockout (KO) would render the cells resistant to that drug, thereby enabling the depletion of non-modified cells and purification of the engineered cells in a cell population. Several monogenic inborn errors of metabolism can be treated by supply of a metabolite and can therefore be seen as models of human auxotrophy.

In certain embodiments, auxotrophy is introduced to human cells by disrupting UMPS in the de novo pyrimidine synthesis pathway through genome editing. This makes the cell's function dependent on the presence of exogenous uridine. Furthermore, this abolishes the cell's ability to metabolize 5-fluoroorotic acid into 5-FU, which enables the depletion of remaining cells with intact UMPS alleles. The ability to use a metabolite to influence the function of human cells by genetically engineered auxotrophy and to deplete other cells provides for the development of this approach for a range of applications where a pure population of controllable cells is necessary.

One example is hereditary orotic aciduria, in which mutations in the UMPS gene lead to a dysfunction that can be treated by supplementation with high doses of uridine (See, Fallon et al (1964). N. Engl. J. Med. 270, 878-881, which is hereby incorporated by reference in its entirety). Transferring this concept to a cell type of interest, genetic engineering is used to knock out the UMPS gene in human cells which makes the cells auxotrophic to uridine and resistant to 5-fluoroorotic acid (5-FOA). We show that UMPS^{-/-} cell lines and primary cells survive and proliferate only in the presence of uridine in vitro, and that UMPS engineered cell proliferation is inhibited without supplementation of uridine in vivo. Furthermore, the cells can be selected from a mixed population by culturing in the presence of 5-FOA.

In certain embodiments, auxotrophy is introduced to human cells by disrupting UMPS in the de-novo pyrimidine synthesis pathway through genome editing. This makes the cell's function dependent on the presence of exogenous uridine. Furthermore, this abolishes the cell's ability to metabolize 5-fluoroorotic acid into 5-FU, which enables the depletion of remaining cells with intact UMPS alleles. The ability to use a metabolite to influence the function of human cells by genetically engineered auxotrophy and to deplete other cells provides for the development of this approach for a range of applications where a pure population of controllable cells is necessary.

One example of an auxotrophy is hereditary orotic aciduria, in which mutations in the UMPS gene lead to a dysfunction that can be treated by supplementation with high doses of uridine (Fallon et al., 1964). Transferring this concept to a cell type of interest, genetic engineering was used to knock out the UMPS gene in human cells which makes the cells auxotrophic to uridine and resistant to 5-fluoroorotic acid (5-FOA). UMPS^{-/-} cell lines and primary cells are shown herein to survive and proliferate only in the presence of uridine in vitro, and that UMPS engineered cell proliferation is inhibited without supplementation of uridine in vivo. Furthermore, the cells can be selected from a mixed population by culturing in the presence of 5-FOA.

### II. Compositions and Methods of Use of Certain Embodiments

Disclosed herein are some embodiments of methods and compositions for use in gene therapy. In some instances, the methods comprise delivery of a transgene, encoding a therapeutic factor, to host cells in a manner that renders the modified host cell auxotrophic, and that can provide improved efficacy, potency, and/or safety of gene therapy through transgene expression. Delivery of the transgene to a specific auxotrophy-inducing locus creates an auxotrophic cell, for example, through disruption or knockout of a gene or downregulation of a gene's activity, that is now dependent on continuous administration of an auxotrophic factor for growth and reproduction. In some instances, the methods comprise nuclease systems targeting the auxotrophy-inducing locus, donor templates or vectors for inserting the transgene, kits, and methods of using such systems, templates or vectors to produce modified cells that are auxotrophic and capable of expressing the introduced transgene.

Also disclosed herein, in some embodiments, are methods, compositions and kits for use of the modified host cells, including pharmaceutical compositions, therapeutic methods, and methods of administration of auxotrophic factors to control—increase, decrease or cease—the growth and reproduction of the modified cells and to control the expression of the transgene and to control levels of the therapeutic factor.

In some instances, delivery of the transgene to the desired locus can be accomplished through methods such as homologous recombination. As used herein, “homologous recombination (HR)” refers to insertion of a nucleotide sequence during repair of double-strand breaks in DNA via homology-directed repair mechanisms. This process uses a “donor” molecule or “donor template” with homology to nucleotide sequence in the region of the break as a template for repairing a double-strand break. The presence of a double-stranded break facilitates integration of the donor sequence. The donor sequence may be physically integrated or used as a template for repair of the break via homologous recombination, resulting in the introduction of all or part of the nucleotide sequence. This process is used by a number of different gene editing platforms that create the double-strand break, such as meganucleases, such as zinc finger nucleases (ZFNs), transcription activator-like effector nucleases (TALENs), and the CRISPR-Cas9 gene editing systems.

In some embodiments, genes are delivered to two or more loci, for example, for the expression of multiple therapeutic factors, or for the introduction of a second gene that acts as a synthetic regulator or that acts to bias the modified cells towards a certain lineage (e.g. by expressing a transcription factor from the second locus). In some embodiments, genes are delivered to two or more auxotrophy-inducing loci. For example, a different gene or a second copy of the same gene is delivered to a second auxotrophy-inducing locus.

In some embodiments, the cell is auxotrophic because the cell no longer has the ability to produce the auxotrophic factor. As used herein, a “cell”, “modified cell” or “modified host cell” refers to a population of cells descended from the same cell, with each cell of the population having a similar genetic make-up and retaining the same modification.

In some embodiments, the auxotrophic factor comprises one or two or more nutrients, enzymes, altered pH, altered temperature, non-organic molecules, non-essential amino acids, or altered concentrations of a moiety (compared to normal physiologic concentrations), or combinations thereof. All references to auxotrophic factor herein contemplate administration of multiple factors. In any of the embodiments described herein, the auxotrophic factor is a nutrient or enzyme that is neither toxic nor bioavailable in the subject in concentrations sufficient to sustain the modified host cell, and it is to be understood that any references to “auxotrophic factor” throughout this application may include reference to a nutrient or enzyme.

In some instances, if the modified cell is not continuously supplied with the auxotrophic factor, the cell ceases proliferation or dies. In some instances, the modified cell provides a safety switch that decreases the risks associated with other cell-based therapies that include oncogenic transformation.

The methods and compositions disclosed herein provide a number of advantages, for example: consistent results and conditions due to integrating into the same locus rather than random integration such as with lentivectors; constant expression of transgene because areas with native promoters or enhancers or areas that are silenced are avoided; a consistent copy number of integration, 1 or 2 copies, rather than a Poisson distribution; and limited chance of oncogenic transformation. In some instances, the modified cells of the present disclosure are less heterogeneous than a product engineered by lentivector or other viral vector.

In some embodiments, disclosed herein, are counter selection methods to generate a population of cells which are 100% auxotrophic, limiting the probability of reversion to a non-auxotrophic state. Current safety switches rely on inserting a transgene, and modified cells can escape through mutation of the transgene or epigenetic silencing of its expression (see, e.g., Wu et al., Mol Ther Methods Clin Dev. 1:14053 (2014), which is hereby incorporated by reference in its entirety). Thus, the combination of transgene insertion with creation of an auxotrophic mechanism is generally safer in the long term.

In some embodiments, reducing the auxotrophic factor administration to low levels may cause the modified cells to enter a quiescent state rather than being killed, permitting temporary interruption and re-starting of therapy with cells already present in the host. This would be an advantage compared to having to re-edit host cells and re-introduce modified host cells.

In some embodiments, ceasing auxotrophic factor administration will result in death of the modified cells when that is desired, for example if aberrant proliferation or oncogenic transformation has been detected, or if cessation of treatment is desired.

In some embodiments, increasing auxotrophic factor administration increases growth and reproduction of the modified cells and results in increased expression of the transgene, and thus increased levels of the therapeutic factor. In some instances, the auxotrophic factor administration provides a means for controlling dosage of the gene product.

An auxotrophy-based safety mechanism circumvents many of the risks to patients associated with current cell therapies. By supplementing a patient with a defined auxotrophic factor during the course of the therapy and removing the factor upon therapy cessation or some other safety-based indication, cell growth is physically limited. In some instances, if the auxotrophic factor is no longer available to the cell, then the cell stops dividing and does not have a self-evident mechanism for the development of resistance. By manipulating levels of the auxotrophic factor, the growth rate of cells in vivo is controlled. Multiple cell lines may be controlled independently in vivo by using separate auxotrophies. Location specific growth may be controlled by localized nutrient release, such as exogenously grown pancreatic B cells administered within a biocompatible device that releases a nutrient and prevents cell escape. For example, the methods and compositions disclosed herein may be used in conjunction with chimeric antigen receptor (CAR)-T cell technology, to allow more defined control over the activity of CAR-T cells in vivo. In some instances, the compositions disclosed herein are used to inhibit or reduce tumor growth. For example, withdrawal of the auxotrophic factor (e.g. uridine or biotin) may lead to tumor regression.

A considerable number of disorders are either caused by an insufficiency of a gene product or are treatable by increased expression of a therapeutic factor, e.g. protein, peptide, antibody, or RNA. In some embodiments, disclosed herein, are compositions comprising modified host cell comprising a transgene encoding a therapeutic factor of interest integrated at an auxotrophy-inducing locus, wherein the modified host cell is auxotrophic for an auxotrophic factor. Further disclosed herein, in some embodiments, are methods of using the compositions of the current disclosure to treat conditions in an individual in need thereof by providing the auxotrophic factor in an amount sufficient to produce therapeutic expression of the factor.

### Exemplary Therapeutic Factors

The following embodiments provide conditions to be treated by producing a therapeutic factor in an auxotrophic host cell.

Clotting disorders, for example, are fairly common genetic disorders where factors in the clotting cascade are absent or have reduced function due to a mutation. These include hemophilia A (factor VIII deficiency), hemophilia B (factor IX deficiency), or hemophilia C (factor XI deficiency).

Alpha-1 antitrypsin (A1AT) deficiency is an autosomal recessive disease caused by defective production of alpha 1-antitrypsin which leads to inadequate A1AT levels in the blood and lungs. It can be associated with the development of chronic obstructive pulmonary disease (COPD) and liver disorders.

Type I diabetes is a disorder in which immune-mediated destruction of pancreatic beta cells results in a profound deficiency of insulin production. Complications include ischemic heart disease (angina and myocardial infarction), stroke and peripheral vascular disease, diabetic retinopathy, diabetic neuropathy, and diabetic nephropathy, which may result in chronic kidney disease requiring dialysis.

Antibodies are secreted protein products used for neutralization or clearance of target proteins that cause disease as well as highly selective killing of certain types of cells (e.g. cancer cells, certain immune cells in autoimmune diseases, cells infected with virus such as human immunodeficiency virus (HIV), RSV, Flu, Ebola, CMV, and others). Antibody therapy has been widely applied to many human conditions including oncology, rheumatology, transplant, and ocular disease. In some instances, the therapeutic factor encoded by the compositions disclosed herein is an antibody used to prevent or treat conditions such as cancer, infectious diseases and autoimmune diseases. In certain embodiments, the cancer is treated by reducing the rate of growth of a tumor or by reducing the size of a tumor in the subject.

Monoclonal antibodies approved by the FDA for therapeutic use include Adalimumab, Bezlotoxumab, Avelumab, Dupilumab, Durvalumab, Ocrelizumab, Brodalumab, Reslizumab, Olaratumab, Daratumumab, Elotuzumab, Necitumumab, Infliximab, Obiltoxaximab, Atezolizumab, Secukinumab, Mepolizumab, Nivolumab, Alirocumab, Idarucizumab, Evolocumab, Dinutuximab, Bevacizumab, Pembrolizumab, Ramucirumab, Vedolizumab, Siltuximab, Alemtuzumab, Trastuzumab emtansine, Pertuzumab, Infliximab, Obinutuzumab, Brentuximab, Raxibacumab, Belimumab, Ipilimumab, Denosumab, Denosumab, Ofatumumab, Besilesomab, Tocilizumab, Canakinumab, Golimumab, Ustekinumab, Certolizumab pegol, Catumaxomab, Eculizumab, Ranibizumab, Panitumumab, Natalizumab, Catumaxomab, Bevacizumab, Omalizumab, Cetuximab, Efalizumab, Ibritumomab tiuxetan, Fanolesomab, Adalimumab, Tositumomab, Alemtuzumab, Trastuzumab, Gemtuzumab ozogamicin, Infliximab, Palivizumab, Necitumumab, Basiliximab, Rituximab, Votumumab, Sulesomab, Arcitumomab, Imiciromab, Capromab, Nofetumomab, Abciximab, Satumomab, and Muromonab-CD3. Bispecific antibody approved by the FDA for therapeutic use includes Blinatumomab. In some embodiments, the antibody is used to prevent or treat HIV or other infectious diseases. Antibodies for use in treatment of HIV include human monoclonal antibody (mAb) VRC-HIVMAB060-00-AB (VRC01); mAb VRC-HIVMAB080-00-AB (VRC01LS); mAb VRC-HIVMAB075-00-AB (VRC07-523LS); mAb F105; mAb C2F5; mAb C2G12; mAb C4E10; antibody UB-421 (targeting the HIV-1 receptor on the CD4 molecule (domain 1) of T-lymphocytes and monocytes); Ccr5mab004 (Human Monoclonal IgG4 antibody to Ccr5); mAb PGDM1400; mAb PGT121 (recombinant human IgG1 monoclonal antibodies that target a V1V2 (PGDM1400) and a V3 glycan-dependent (PGT121) epitope region of the HIV envelope protein); KD-247 (a humanized monoclonal antibody); PRO 140 (a monoclonal CCR5 antibody); mAb 3BNC117; and PG9 (anti-HIV-1 gp120 monoclonal antibody).

Therapeutic RNAs include antisense, siRNAs, aptamers, microRNA mimics/anti-miRs and synthetic mRNA, and some of these can be expressed by transgenes.

LSDs are inherited metabolic diseases that are characterized by an abnormal build-up of various toxic materials in the body's cells as a result of enzyme deficiencies. There are nearly 50 of these disorders altogether, and they affect different parts of the body, including the skeleton, brain, skin, heart, and central nervous system. Common examples include Sphingolipidoses, Farber disease (ASAH1 deficiency), Krabbe disease (galactosylceramidase or GALC deficiency), Galactosialidosis, Gangliosidoses, Alpha-galactosidase, Fabry disease (α-galactosidase deficiency—GLA, or agalsidase alpha/beta), Schindler disease (alpha-NAGA deficiency), GM1 gangliosidosis, GM2 gangliosidoses (beta-hexosaminidase deficiency), Sandhoff disease (hexosaminidase-B deficiency), Tay-Sachs disease (hexosaminidase-A deficiency), Gaucher's disease Type 1/2/3 (glucocerebrosidase deficiency-gene name: GBA), Wolman disease (LAL deficiency), Niemann-Pick disease type AB (sphingomyelin phosphodiesterase 1deficiency—SMPD1 or acid sphingomyelinase), Sulfatidosis, Metachromatic leukodystrophy, Hurler syndrome (alpha-L iduronidase deficiency—IDUA), Hunter syndrome or MPS2 (iduronate-2-sulfatase deficiency-idursulfase or IDS), Sanfilippo syndrome, Morquio, Maroteaux-Lamy syndrome, Sly syndrome (β-glucuronidase deficiency), Mucolipidosis, I-cell disease, Lipidosis, =Neuronal ceroid lipofuscinoses, Batten disease (tripeptidyl peptidase-1 deficiency), Pompe (alglucosidase alpha deficiency), hypophosphatasia (asfotase alpha deficiency), MPS1 (laronidase deficiency), MPS3A (heparin N-sulfatase deficiency), MPS3B (alpha-N-acetylglucosaminidase deficiency), MPS3C (heparin-a-glucosaminide N-acetyltransferase deficiency), MPS3D (N-acetylglucosamine 6-sulfatase deficiency), MPS4 (elosulfase alpha deficiency), MPS6 (glasulfate deficiency), MPS7 (B-glucoronidase deficiency), phenylketonuria (phenylalanine hydroxylase deficiency), and MLD (arylsulphatase A deficiency). Collectively LSDs have an incidence in the population of about 1 in 7000 births and have severe effects including early death. While clinical trials are in progress on possible treatments for some of these diseases, there is currently no approved treatment for many LSDs. Current treatment options for some but not all LSDs include enzyme replacement therapy (ERT). ERT is a medical treatment which replaces an enzyme that is deficient or absent in the body. In some instances, this is done by giving the patient an intravenous (IV) infusion of a solution containing the enzyme.

Disclosed herein, in some embodiments, are methods of treating a LSD in an individual in need thereof, the method comprising providing to the individual enzyme replacement therapy using the compositions disclosed herein. In some instances, the method comprises a modified host cell ex vivo, comprising a transgene encoding an enzyme integrated at an auxotrophy-inducing locus, wherein said modified host cell is auxotrophic for an auxotrophic factor and capable of expressing the enzyme that is deficient in the individual, thereby treating the LSD in the individual. In some instances, the auxotrophy-inducing locus is within a gene in Table 1 or within a region that controls expression of a gene in Table 1. In some instances, the auxotrophy-inducing locus is within a gene encoding uridine monophosphate synthetase (UMPS). In some instances, the auxotrophic factor is uridine. In some instances, the auxotrophy-inducing locus is within a gene encoding holocarboxylase synthetase (HLCS). In some instances, the auxotrophic factor is biotin. In some instances, the auxotrophy-inducing locus is within a gene encoding asparagine synthetase. In some instances, the auxotrophic factor is asparagine. In some instances, the auxotrophy-inducing locus is within a gene encoding aspartate transaminase. In some instances, the auxotrophic factor is aspartate. In some instances, the auxotrophy-inducing locus is within a gene encoding alanine transaminase. In some instances, the auxotrophic factor is alanine. In some instances, the auxotrophy-inducing locus is within a gene encoding cystathionine beta synthase. In some instances, the auxotrophic factor is cysteine. In some instances, the auxotrophy-inducing locus is within a gene encoding cystathionine gamma-lyase. In some instances, the auxotrophic factor is cysteine. In some instances, the auxotrophy-inducing locus is within a gene encoding glutamine synthetase. In some instances, the auxotrophic factor is glutamine. In some instances, the auxotrophy-inducing locus is within a gene encoding serine hydroxymethyltransferase. In some instances, the auxotrophic factor is serine or glycine. In some instances, the auxotrophy-inducing locus is within a gene encoding glycine synthase. In some instances, the auxotrophic factor is glycine. In some instances, the auxotrophy-inducing locus is within a gene encoding phosphoserine transaminase. In some instances, the auxotrophic factor is serine. In some instances, the auxotrophy-inducing locus is within a gene encoding phosphoserine phosphatase. In some instances, the auxotrophic factor is serine. In some instances, the auxotrophy-inducing locus is within a gene encoding phenylalanine hydroxylase. In some instances, the auxotrophic factor is tyrosine. In some instances, the auxotrophy-inducing locus is within a gene encoding argininosuccinate synthetase. In some instances, the auxotrophic factor is arginine. In some instances, the auxotrophy-inducing locus is within a gene encoding argininosuccinate lyase. In some instances, the auxotrophic factor is arginine. In some instances, the auxotrophy-inducing locus is within a gene encoding dihydrofolate reductase. In some instances, the auxotrophic factor is folate or tetrahydrofolate.

Further disclosed herein, in some embodiments, are methods of treating a disease or disorder in an individual in need thereof, the method comprising providing to the individual protein replacement therapy using the compositions disclosed herein. In some instances, the method comprises a modified host cell ex vivo, comprising a transgene encoding a protein integrated at an auxotrophy-inducing locus, wherein said modified host cell is auxotrophic for an auxotrophic factor and capable of expressing the protein that is deficient in the individual, thereby treating the disease or disorder in the individual. In some instances, the auxotrophy-inducing locus is within a gene in Table 1 or within a region that controls expression of a gene in Table 1. In some instances, the auxotrophy-inducing locus is within a gene encoding uridine monophosphate synthetase (UMPS). In some instances, the auxotrophic factor is uridine. In some instances, the auxotrophy-inducing locus is within a gene encoding holocarboxylase synthetase (HLCS). In some instances, the auxotrophic factor is biotin. In some instances, the disease is Friedreich's ataxia, and the protein is frataxin. In some instances, the disease is hereditary angioedema and the protein is C1 esterase inhibitor (e.g., HAEGAARDA® subcutaneous injection). In some instances, the disease is spinal muscular atrophy and the protein is SMN1.

### III. Compositions and Methods for Making Modified Cells

A. Cells

Disclosed herein, in some embodiments, are compositions comprising modified host cells, preferably human cells, that are genetically engineered to be auxotrophic (through insertion of a transgene encoding a therapeutic factor at an auxotrophy-inducing locus) and are capable of expressing the therapeutic factor. Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, the cell is an embryonic stem cell, a stem cell, a progenitor cell, a pluripotent stem cell, an induced pluripotent stem (iPS) cell, a somatic stem cell, a differentiated cell, a mesenchymal stem cell or a mesenchymal stromal cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell, or a peripheral blood mononuclear cell (PBMC). For example, the cell may be engineered to express a CAR, thereby creating a CAR-T cell. In some embodiments, the cell lines are T cells that are genetically engineered to be auxotrophic. Engineered auxotrophic T cells may be administered to a patient with cancer along with an auxotrophic factor. Upon destruction of the cancer, the auxotrophic nutrient may be removed, which results in the elimination of the engineered auxotrophic T cells. In some embodiments, the cell lines are pluripotent stem cells that are genetically engineered to be auxotrophic. Engineered auxotrophic pluripotent stem cells may be administered to a patient along with an auxotrophic factor. Upon conversion of an engineered auxotrophic pluripotent stem cell to a cancerous cell, the auxotrophic factor may be removed, which results in the elimination of the cancerous cell and the engineered auxotrophic pluripotent stem cells.

To prevent immune rejection of the modified cells when administered to a subject, the cells to be modified are preferably derived from the subject's own cells. Thus, preferably the mammalian cells are from the subject to be treated with the modified cells. In some instances, the mammalian cells are modified to be autologous cell. In some instances, the mammalian cells are further modified to be allogeneic cell. In some instances, modified T cells can be further modified to be allogeneic, for example, by inactivating the T cell receptor locus. In some instances, modified cells can further be modified to be allogeneic, for example, by deleting B2M to remove MEW class I on the surface of the cell, or by deleting B2M and then adding back an HLA-G-B2M fusion to the surface to prevent NK cell rejection of cells that do not have MEW Class I on their surface.

The cell lines may include stem cells that were maintained and differentiated using the techniques below as shown in U.S. Pat. No. 8,945,862, which is hereby incorporated by reference in its entirety. In some embodiments, the stem cell is not a human embryonic stem cell. Furthermore, the cell lines may include stem cells made by the techniques disclosed in WO 2003/046141 or Chung et al. (Cell Stem Cell, February 2008, Vol. 2, pages 113-117); each of which are hereby incorporated by reference in its entirety.

For example, the cells may be stem cells isolated from the subject for use in a regenerative medical treatment in any of epithelium, cartilage, bone, smooth muscle, striated muscle, neural epithelium, stratified squamous epithelium, and ganglia. Disease that results from the death or dysfunction of one or a few cell types, such as Parkinson's disease and juvenile onset diabetes, are also commonly treated using stem cells (See, Thomson et al., Science, 282:1145-1147, 1998, which is hereby incorporated by reference in its entirety).

In some embodiments, cells are harvested from the subject and modified according to the methods disclosed herein, which can include selecting certain cell types, optionally expanding the cells and optionally culturing the cells, and which can additionally include selecting cells that contain the transgene integrated into the auxotrophy-inducing locus.

B. Donor Templates or Vectors for Inserting the Transgene

In some embodiments, the compositions disclosed herein comprise donor templates or vectors for inserting the transgene into the auxotrophy-inducing locus.

In some embodiments, the donor template comprises (a) one or more nucleotide sequences homologous to a fragment of the auxotrophy-inducing locus, or homologous to the complement of said auxotrophy-inducing locus, and (b) a transgene encoding a therapeutic factor, optionally linked to an expression control sequence. For example, after a nuclease system is used to cleave DNA, introduction of a donor template can take advantage of homology-directed repair mechanisms to insert the transgene sequence during their repair of the break in the DNA. In some instances, the donor template comprises a region that is homologous to nucleotide sequence in the region of the break so that the donor template hybridizes to the region adjacent to the break and is used as a template for repairing the break.

In some embodiments, the transgene is flanked on both sides by nucleotide sequences homologous to a fragment of the auxotrophy-inducing locus or the complement thereof.

In some instances, the donor template is single stranded, double stranded, a plasmid or a DNA fragment.

In some instances, plasmids comprise elements necessary for replication, including a promoter and optionally a 3′ UTR.

Further disclosed herein are vectors comprising (a) one or more nucleotide sequences homologous to a fragment of the auxotrophy-inducing locus, or homologous to the complement of said auxotrophy-inducing locus, and (b) a transgene encoding a therapeutic factor.

The vector can be a viral vector, such as a retroviral, lentiviral (both integration competent and integration defective lentiviral vectors), adenoviral, adeno-associated viral or herpes simplex viral vector. Viral vectors may further comprise genes necessary for replication of the viral vector.

In some embodiments, the targeting construct comprises: (1) a viral vector backbone, e.g. an AAV backbone, to generate virus; (2) arms of homology to the target site of at least 200 bp but ideally 400 bp on each side to assure high levels of reproducible targeting to the site (see, Porteus, Annual Review of Pharmacology and Toxicology, Vol. 56:163-190 (2016); which is hereby incorporated by reference in its entirety); (3) a transgene encoding a therapeutic factor and capable of expressing the therapeutic factor; (4) an expression control sequence operably linked to the transgene; and optionally (5) an additional marker gene to allow for enrichment and/or monitoring of the modified host cells.

Suitable marker genes are known in the art and include Myc, HA, FLAG, GFP, truncated NGFR, truncated EGFR, truncated CD20, truncated CD19, as well as antibiotic resistance genes.

Any AAV known in the art can be used. In some embodiments the primary AAV serotype is AAV6.

In any of the preceding embodiments, the donor template or vector comprises a nucleotide sequence homologous to a fragment of the auxotrophy-inducing locus, optionally any of the genes in Table 1 below, wherein the nucleotide sequence is at least 85, 88, 90, 92, 95, 98, or 99% identical to at least 200, 250, 300, 350, or 400 consecutive nucleotides of the auxotrophy-inducing locus; up to 400 nucleotides is usually sufficient to assure accurate recombination. Any combination of the foregoing parameters is envisioned, e.g. at least 85% identical to at least 200 consecutive nucleotides, or at least 88% identical to at least 200 consecutive nucleotides, or at least 90% identical to at least 200 consecutive nucleotides, or at least 92% identical to at least 200 consecutive nucleotides, or at least 95% identical to at least 200 consecutive nucleotides, or at least 98% identical to at least 200 consecutive nucleotides, or at least 99% identical to at least 200 consecutive nucleotides, or at least 85% identical to at least 250 consecutive nucleotides, or at least 88% identical to at least 250 consecutive nucleotides, or at least 90% identical to at least 250 consecutive nucleotides, or at least 92% identical to at least 250 consecutive nucleotides, or at least 95% identical to at least 250 consecutive nucleotides, or at least 98% identical to at least 250 consecutive nucleotides, or at least 99% identical to at least 250 consecutive nucleotides, or at least 85% identical to at least 300 consecutive nucleotides, or at least 88% identical to at least 300 consecutive nucleotides, or at least 90% identical to at least 300 consecutive nucleotides, or at least 92% identical to at least 300 consecutive nucleotides, or at least 95% identical to at least 300 consecutive nucleotides, or at least 98% identical to at least 300 consecutive nucleotides, or at least 99% identical to at least 300 consecutive nucleotides, or at least 85% identical to at least 350 consecutive nucleotides, or at least 88% identical to at least 350 consecutive nucleotides, or at least 90% identical to at least 350 consecutive nucleotides, or at least 92% identical to at least 350 consecutive nucleotides, or at least 95% identical to at least 350 consecutive nucleotides, or at least 98% identical to at least 350 consecutive nucleotides, or at least 99% identical to at least 350 consecutive nucleotides, or at least 85% identical to at least 400 consecutive nucleotides, or at least 88% identical to at least 400 consecutive nucleotides, or at least 90% identical to at least 400 consecutive nucleotides, or at least 92% identical to at least 400 consecutive nucleotides, or at least 95% identical to at least 400 consecutive nucleotides, or at least 98% identical to at least 400 consecutive nucleotides, or at least 99% identical to at least 400 consecutive nucleotides.

The disclosure herein also contemplates a system for targeting integration of a transgene to an auxotrophy-inducing locus comprising said donor template or vector, a cas9 protein, and a guide RNA.

The disclosure herein further contemplates a system for targeting integration of a transgene to an auxotrophy-inducing locus comprising said donor template or vector and a meganuclease specific for said auxotrophy-inducing locus. The meganuclease can be, for example, a ZFN or TALEN.

The inserted construct can also include other safety switches, such as a standard suicide gene into the locus (e.g. iCasp9) in circumstances where rapid removal of cells might be required due to acute toxicity. The present disclosure provides a robust safety switch so that any engineered cell transplanted into a body can be eliminated by removal of an auxotrophic factor. This is especially important if the engineered cell has transformed into a cancerous cell.

In some instances, the donor polynucleotide or vector optionally further comprises an expression control sequence operably linked to said transgene. In some embodiments, the expression control sequence is a promoter or enhancer, an inducible promoter, a constitutive promoter, a tissue-specific promoter or expression control sequence, a posttranscriptional regulatory sequence or a microRNA.

C. Nuclease Systems

In some embodiments, the compositions disclosed herein comprise nuclease systems targeting the auxotrophy-inducing locus. For example, the present disclosure contemplates (a) a meganuclease that targets and cleaves DNA at said auxotrophy-inducing locus, or (b) a polynucleotide that encodes said meganuclease, including a vector system for expressing said meganuclease. As one example, the meganuclease is a TALEN that is a fusion protein comprising (i) a Transcription Activator Like Effector (TALE) DNA binding domain that binds to the auxotrophy-inducing locus, wherein the TALE DNA binding protein comprises a plurality of TALE repeat units, each TALE repeat unit comprising an amino acid sequence that binds to a nucleotide in a target sequence in the auxotrophy-inducing locus, and (ii) a DNA cleavage domain.

Also disclosed herein are CRISPR/Cas or CRISPR/Cpf1 system that targets and cleaves DNA at said auxotrophy-inducing locus that comprises (a) a Cas (e.g. Cas9) or Cpf1 polypeptide or a nucleic acid encoding said polypeptide, and (b) a guide RNA that hybridizes specifically to said auxotrophy-inducing locus, or a nucleic acid encoding said guide RNA. In nature, the Cas9 system is composed of a cas9 polypeptide, a crRNA, and a trans-activating crRNA (tracrRNA). As used herein, “cas9 polypeptide” refers to a naturally occurring cas9 polypeptide or a modified cas9 polypeptide that retains the ability to cleave at least one strand of DNA. The modified cas9 polypeptide can, for example, be at least 75%, 80%, 85%, 90%, or 95% identical to a naturally occurring Cas9 polypeptide. Cas9 polypeptides from different bacterial species can be used; S. pyogenes is commonly sold commercially. The cas9 polypeptide normally creates double-strand breaks but can be converted into a nickase that cleaves only a single strand of DNA (i.e. produces a “single stranded break”) by introducing an inactivating mutation into the HNH or RuvC domain. Similarly, the naturally occurring tracrRNA and crRNA can be modified as long as they continue to hybridize and retain the ability to target the desired DNA, and the ability to bind the cas9. The guide RNA can be a chimeric RNA, in which the two RNAs are fused together, e.g. with an artificial loop, or the guide RNA can comprise two hybridized RNAs. The meganuclease or CRISPR/Cas or CRISPR/Cpf1 system can produce a double stranded break or one or more single stranded breaks within the auxotrophy-inducing locus, for example, to produce a cleaved end that includes an overhang.

In some instances, the nuclease systems described herein, further comprises a donor template as described herein.

Various methods are known in the art for editing nucleic acid, for example to cause a gene knockout or expression of a gene to be downregulated. For example, various nuclease systems, such as zinc finger nucleases (ZFN), transcription activator-like effector nucleases (TALEN), meganucleases, or combinations thereof are known in the art to be used to edit nucleic acid and may be used in the present disclosure. Meganucleases are modified versions of naturally occurring restriction enzymes that typically have extended or fused DNA recognition sequences.

The CRISPR/Cas system is detailed in, for example WO 2013/176772, WO 2014/093635 and WO 2014/089290; each of which is hereby incorporated by reference in its entirety. Its use in T cells is suggested in WO 2014/191518, which is hereby incorporated by reference in its entirety. CRISPR engineering of T cells is discussed in EP 3004349, which is hereby incorporated by reference in its entirety.

The time-limiting factor for generation of mutant (knock-out, knock-in, or gene replaced) cell lines was the clone screening and selection before development of the CRISPR/Cas9 platform. The term “CRISPR/Cas9 nuclease system” as used herein, refers to a genetic engineering tool that includes a guide RNA (gRNA) sequence with a binding site for Cas9 and a targeting sequence specific for the area to be modified. The Cas9 binds the gRNA to form a ribonucleoprotein that binds and cleaves the target area. CRISPR/Cas9 permits easy multiplexing of multiple gene edits. In some embodiments, the gRNA comprises the nucleic acid sequence of SEQ ID NO: 1.

In addition to the CRISPR/Cas 9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used in methods to generate the cell lines disclosed herein. For example, the CRISPR system used may be the CRISPR/Cas9 system, such as the S. pyogenes CRISPR/Cas9 system.

### IV. Methods of Creating the Modified Host Cells

In some embodiments, the auxotrophy-inducing locus is within a target gene selected from those disclosed in Table 1, or the region controlling expression of that gene. In some embodiments, the target gene is selected from UMPS (creating a cell line auxotrophic for uracil) and holocarboxylase synthetase (creating a cell line auxotrophic for biotin). In some embodiments, the auxotrophic factor is selected from biotin, alanine, aspartate, asparagine, glutamate, serine, uracil and cholesterol.

Further disclosed herein are methods of using said nuclease systems to produce the modified host cells described herein, comprising introducing into the cell (a) the components of one or more nuclease systems that target and cleave DNA at an auxotrophy-inducing locus, e.g. meganuclease such as ZFN or TALEN, or CRISPR/Cas nuclease such as CRISPR/Cas9, and (b) a donor template or vector as described herein. Each component can be introduced into the cell directly or can be expressed in the cell by introducing a nucleic acid encoding the components of said one or more nuclease systems. The methods can also comprise introducing a second nuclease system, e.g. a second meganuclease or second CRISPR/Cas nuclease that targets and cleaves DNA at a second locus, or a second guide RNA that targets DNA at a second locus, or a nucleic acid that encodes any of the foregoing, and (b) a second donor template or vector. The second donor template or vector can contain a different transgene, or a second copy of the same transgene, which will then be integrated at the second locus according to such methods.

Such methods will target integration of the transgene encoding the therapeutic factor to an auxotrophy-inducing locus in a host cell ex vivo.

Such methods can further comprise (a) introducing a donor template or vector into the cell, optionally after expanding said cells, or optionally before expanding said cells, and (b) optionally culturing the cell.

In some embodiments, the disclosure herein contemplates a method of producing a modified mammalian host cell comprising introducing into a mammalian cell: (a) a Cas9 polypeptide, or a nucleic acid encoding said Cas9 polypeptide, (b) a guide RNA specific to an auxotrophy-inducing locus, or a nucleic acid encoding said guide RNA, and (c) a donor template or vector as described herein. The methods can also comprise introducing (a) a second guide RNA specific to a second auxotrophy-inducing locus and (b) a second donor template or vector. In such methods, the guide RNA can be a chimeric RNA or two hybridized RNAs.

In any of these methods, the nuclease can produce one or more single stranded breaks within the auxotrophy-inducing locus, or a double stranded break within the auxotrophy-inducing locus. In these methods, the auxotrophy-inducing locus is modified by homologous recombination with said donor template or vector to result in insertion of the transgene into the locus.

The methods can further comprise (c) selecting cells that contain the transgene integrated into the auxotrophy-inducing locus. The selecting steps can include (i) selecting cells that require the auxotrophic factor to survive and optionally (ii) selecting cells that comprise the transgene integrated into the auxotrophy-inducing locus.

In some embodiments, the auxotrophy-inducing locus is a gene encoding uridine monophosphate synthetase and the cells are selected by contacting them with 5-FOA. The UMPS gene is required to metabolize 5-FOA into 5-FUMP, which is toxic to cells due to its incorporation into RNA/DNA. Thus, cells which have a disruption in the UMPS gene will survive 5-FOA treatment. The resulting cells will all be auxotrophic, although not all cells may contain the transgene. Subsequent positive selection for the transgene will isolate only modified host cells that are auxotrophic and that are also capable of expressing the transgene.

In some embodiments, the disclosure herein provides a method of creating a modified human host cell comprising the steps of: (a) obtaining a pool of cells, (b) using a nuclease to introduce a transgene to the auxotrophy-inducing locus, for example by knocking out or downregulating expression of a gene, and (c) screening for auxotrophy, and (d) screening for the presence of the transgene.

The screening step may be carried out by culturing the cells with or without one of the auxotrophic factors disclosed in Table 1.

Techniques for insertion of transgenes, including large transgenes, capable of expressing functional factors, antibodies and cell surface receptors are known in the art (See, e.g. Bak and Porteus, Cell Rep. 2017 Jul. 18; 20(3): 750-756 (integration of EGFR); Kanojia et al., Stem Cells. 2015 October; 33(10):2985-94 (expression of anti-Her2 antibody); Eyquem et al., Nature. 2017 Mar. 2; 543(7643):113-117 (site-specific integration of a CAR); O'Connell et al., 2010 PLoS ONE 5(8): e12009 (expression of human IL-7); Tuszynski et al., Nat Med. 2005 May; 11(5):551-5 (expression of NGF in fibroblasts); Sessa et al., Lancet. 2016 Jul. 30; 388(10043):476-87 (expression of arylsulfatase A in ex vivo gene therapy to treat MLD); Rocca et al., Science Translational Medicine 25 Oct. 2017: Vol. 9, Issue 413, eaaj2347 (expression of frataxin); Bak and Porteus, Cell Reports, Vol. 20, Issue 3, 18 Jul. 2017, Pages 750-756 (integrating large transgene cassettes into a single locus), Dever et al., Nature 17 Nov. 2016: 539, 384-389 (adding tNGFR into hematopoietic stem cells (HSC) and HSPCs to select and enrich for modified cells); each of which is hereby incorporated by reference in its entirety.

A. Auxotrophy-Inducing Locus and Auxotrophic Factor

In some embodiments, disruption of a single gene causes the desired auxotrophy. In alternative embodiments, disruption of multiple genes produces the desired auxotrophy.

In some embodiments, the auxotrophy-inducing locus is a gene encoding a protein that produces an auxotrophic factor, which includes proteins upstream in the pathway for producing the auxotrophic factor.

In some embodiments described herein, the auxotrophy-inducing locus is the gene encoding uridine monophosphate synthetase (UMPS) (and the corresponding auxotrophic factor is uracil), or the gene encoding holocarboxylase synthetase (and the corresponding auxotrophic factor is biotin). In some embodiments, auxotrophy-inducing loci are selected from the following genes in Table 1. The genes of Table 1 were collated by selecting S. cerevisiae genes with a phenotype annotated as “Auxotrophy” downloaded with “Chemical” data from the yeast phenotype ontology database on the Saccharomyces genome database (SGD) (See, Cherry et al. 2012, Nucleic Acids Res. 40:D700-D705, which is hereby incorporated by reference in its entirety). These genes were converted into human homologues using the YeastMine® database or, in alternative embodiments, the Saccharomyces Genome Database (SGD). The genes are identified by their ENSEMBL gene symbol and ENSG identifier, which are found in the ENSEMBL database (www.ensembl.org). The first five zeroes of the ENSG identifiers (e.g., ENSG00000) have been removed.

TABLE 1
Auxotrophy-inducing loci

| Gene | ENSG(s) | Auxotrophic factor |
|---|---|---|
| AACS | 081760 | Lysine |
| AADAT | 109576 | histidine |
| AASDHPPT | 149313 | Lysine |
| AASS | 008311 | Lysine |
| ACAT1 | 075239 | ergosterol |
| ACCS | 110455 | histidine |
| ACCSL | 205126 | histidine |
| ACO1 | 122729 | leucine |
| ACO2 | 100412 | leucine |
| ACSS3 | 111058 | Lysine |
| ADSL | 239900 | adenine |
| ADSS | 035687 | adenine |
| ADSSL1 | 185100 | adenine |
| ALAD | 148218 | cysteine |
| ALAS1 | 023330 | cysteine |
| ALAS2 | 158578 | cysteine |
| ALDH1A1 | 165092 | pantothenic acid |
| ALDH1A2 | 128918 | pantothenic acid |
| ALDH1A3 | 184254 | pantothenic acid |
| ALDH1B1 | 137124 | pantothenic acid |
| ALDH2 | 111275 | pantothenic acid |
| AMD1 | 123505 | 0.25 mM spermine |
| ASL | 126522 | arginine |
| ASS1 | 130707 | arginine |
| ATF4 | 128272 | methionine |
| ATF5 | 169136 | methionine |
| AZIN1 | 155096 | 0.25 mM putrescine |
| AZIN2 | 142920 | 0.25 mM putrescine |
| BCAT1 | 060982 | valine, leucine |
| BCAT2 | 105552 | valine, leucine |
| CAD | 084774 | Uracil |
| CBS | 160200 | cysteine |
| CBSL | 274276 | cysteine |
| CCBL1 | 171097 | histidine |
| CCBL2 | 137944 | histidine |
| CCS | 173992 | methionine |
| CEBPA | 245848 | methionine |
| CEBPB | 172216 | methionine |
| CEBPD | 221869 | methionine |
| CEBPE | 092067 | methionine |
| CEBPG | 153879 | methionine |
| CH25H | 138135 | ergosterol |
| COQ6 | 119723 | nicotinic acid |
| CPS1 | 021826 | arginine |
| CTH | 116761 | cysteine |
| CYP51A1 | 001630 | ergosterol |
| DECR1 | 104325 | ergosterol |
| DHFR | 228716 | dTMP |
| DHFRL1 | 178700 | dTMP |
| DHODH | 102967 | Uracil |
| DHRS7 | 100612 | Lysine |
| DHRS7B | 109016 | Lysine |
| DHRS7C | 184544 | Lysine |
| DPYD | 188641 | Uracil |
| DUT | 128951 | dTMP |
| ETFDH | 171503 | thiamine(1+) |
| FAXDC2 | 170271 | ergosterol |
| FDFT1 | 079459; 284967 | ergosterol |
| FDPS | 160752 | ergosterol |
| FDXR | 161513 | Uracil |
| FH | 091483 | arginine |
| FPGS | 136877 | methionine |
| G6PD | 160211 | methionine |
| GCAT | 100116 | cysteine |
| GCH1 | 131979 | 5-formyltetrahydrofolic |
| | | acid |
| GCLC | 001084 | glutathione |
| GFPT1 | 198380 | D-glucosamine |
| GFPT2 | 131459 | D-glucosamine |
| GLRX5 | 182512 | glutamic acid |
| GLUL | 135821 | glutamine |
| GMPS | 163655 | guanine |
| GPT | 167701 | histidine |
| GPT2 | 166123 | histidine |
| GSX2 | 180613 | adenine |
| H6PD | 049239 | methionine |
| HAAO | 162882 | nicotinic acid |
| HLCS | 159267 | biotin |
| HMBS | 256269; 281702 | heme |
| HMGCL | 117305 | lysine |
| HMGCLL1 | 146151 | lysine |
| HMGCS1 | 112972 | ergosterol |
| HMGCS2 | 134240 | ergosterol |
| HOXA1 | 105991 | adenine |
| HOXA10 | 253293 | adenine |
| HOXA11 | 005073 | adenine |
| HOXA13 | 106031 | adenine |
| HOXA2 | 105996 | adenine |
| HOXA3 | 105997 | adenine |
| HOXA4 | 197576 | adenine |
| HOXA5 | 106004 | adenine |
| HOXA6 | 106006 | adenine |
| HOXA7 | 122592 | adenine |
| HOXA9 | 078399 | adenine |
| HOXB1 | 120094 | adenine |
| HOXB13 | 159184 | adenine |
| HOXB2 | 173917 | adenine |
| HOXB3 | 120093 | adenine |
| HOXB4 | 182742 | adenine |
| HOXB5 | 120075 | adenine |
| HOXB6 | 108511 | adenine |
| HOXB7 | 260027 | adenine |
| HOXB8 | 120068 | adenine |
| HOXB9 | 170689 | adenine |
| HOXC10 | 180818 | adenine |
| HOXC11 | 123388 | adenine |
| HOXC12 | 123407 | adenine |
| HOXC13 | 123364 | adenine |
| HOXC4 | 198353 | adenine |
| HOXC5 | 172789 | adenine |
| HOXC6 | 197757 | adenine |
| HOXC8 | 037965 | adenine |
| HOXC9 | 180806 | adenine |
| HOXD1 | 128645 | adenine |
| HOXD10 | 128710 | adenine |
| HOXD11 | 128713 | adenine |
| HOXD12 | 170178 | adenine |
| HOXD13 | 128714 | adenine |
| HOXD3 | 128652 | adenine |
| HOXD4 | 170166 | adenine |
| HOXD8 | 175879 | adenine |
| HOXD9 | 128709 | adenine |
| HRSP12 | 132541 | isoleucine |
| HSD11B1 | 117594 | lysine |
| HSD11B1L | 167733 | lysine |
| HSD17B12 | 149084 | ergosterol |
| HSD17B3 | 130948 | ergosterol |
| HSD17B7 | 132196 | ergosterol |
| HSD17B7P2 | 099251 | ergosterol |
| HSDL1 | 103160 | ergosterol |
| HSDL2 | 119471 | ergosterol |
| IBA57 | 181873 | glutamic acid |
| IDO1 | 131203 | nicotinic acid |
| IDO2 | 188676 | nicotinic acid |
| IL4I1 | 104951 | 0.1 mM beta-alanine |
| ILVBL | 105135 | valine, isoleucine |
| IP6K1 | 176095 | arginine |
| IP6K2 | 068745 | arginine |
| IP6K3 | 161896 | arginine |
| IPMK | 151151 | arginine |
| IREB2 | 136381 | leucine |
| ISCA1 | 135070 | lysine |
| ISCA1P1 | 217416 | lysine |
| ISCA2 | 165898 | lysine |
| KATNA1 | 186625 | ethanolamine |
| KATNAL1 | 102781 | ethanolamine |
| KATNAL2 | 167216 | ethanolamine |
| KDM1B | 165097 | 0.1 mM beta-alanine |
| KDSR | 119537 | lysine |
| KMO | 117009 | nicotinic acid |
| KYNU | 115919 | nicotinic acid |
| LGSN | 146166 | glutamine |
| LSS | 281289; 160285 | ergosterol |
| MARS | 166986 | methionine |
| MARS2 | 247626 | methionine |
| MAX | 125952 | methionine |
| MITF | 187098 | glutamate (1−) |
| MLX | 108788 | glutamate(1−) |
| MMS19 | 155229 | methionine |
| MPC1 | 060762 | valine, leucine |
| MPC1L | 238205 | valine, leucine |
| MPI | 178802 | D-mannose |
| MSMO1 | 052802 | ergosterol |
| MTHFD1 | 100714 | adenine |
| MTHFD1L | 120254 | adenine |
| MTHFD2 | 065911 | adenine |
| MTHFD2L | 163738 | adenine |
| MTHFR | 177000 | methionine |
| MTRR | 124275 | methionine |
| MVK | 110921 | ergosterol |
| MYB | 118513 | adenine |
| MYBL1 | 185697 | adenine |
| MYBL2 | 101057 | adenine |
| NAGS | 161653 | arginine |
| ODC1 | 115758 | 0.25 mM putrescine |
| OTC | 036473 | arginine |
| PAICS | 128050 | adenine |
| PAOX | 148832 | 0.1 mM beta-alanine |
| PAPSS1 | 138801 | methionine |
| PAPSS2 | 198682 | methionine |
| PDHB | 168291 | tryptophan |
| PDX1 | 139515 | adenine |
| PFAS | 178921 | adenine |
| PIN1 | 127445 | galactose |
| PLCB1 | 182621 | omithine |
| PLCB2 | 137841 | ornithine |
| PLCB3 | 149782 | ornithine |
| PLCB4 | 101333 | ornithine |
| PLCD1 | 187091 | ornithine |
| PLCD3 | 161714 | ornithine |
| PLCD4 | 115556 | ornithine |
| PLCE1 | 138193 | ornithine |
| PLCG1 | 124181 | ornithine |
| PLCG2 | 197943 | ornithine |
| PLCH1 | 114805 | ornithine |
| PLCH2 | 276429; 149527 | ornithine |
| PLCL1 | 115896 | ornithine |
| PLCL2 | 154822; 284017 | ornithine |
| PLCZ1 | 139151 | ornithine |
| PM20D1 | 162877 | leucine |
| PPAT | 128059 | adenine |
| PSAT1 | 135069 | serine |
| PSPH | 146733 | serine |
| PYCR1 | 183010 | proline |
| PYCR2 | 143811 | proline |
| | 104524 | proline |
| QPRT | 103485 | Nicotinic acid |
| RDH8 | 80511 | Lysine |
| RPUSD2 | 166133 | riboflavin |
| SCD | 99194 | oleic acid |
| SCD5 | 145284 | oleic acid |
| SLC25A19 | 125454 | thiamine |
| SLC25A26 | 144741; 282739 | biotin |
| SLC25A34 | 162461 | leucine |
| SLC25A35 | 125434 | leucine |
| SLC7A10 | 130876 | L-arginine |
| SLC7A11 | 151012 | L-arginine |
| SLC7A13 | 164893 | L-arginine |
| SLC7A5 | 103257 | L-arginine |
| SLC7A6 | 103064 | L-arginine |
| SLC7A7 | 155465 | L-arginine |
| SLC7A8 | 092068 | L-arginine |
| SLC7A9 | 021488 | L-arginine |
| SMOX | 088826 | 0.1 mM beta-alanine |
| SMS | 102172 | 0.25 mM spermine |
| SNAPC4 | 165684 | adenine |
| SOD1 | 142168 | methionine |
| SOD3 | 109610 | methionine |
| SQLE | 104549 | ergosterol |
| SRM | 116649 | 0.25 mM spermine |
| TAT | 198650 | histidine |
| TFE3 | 068323 | glutamate(1−) |
| TFEB | 112561 | glutamate(1−) |
| TFEC | 105967 | glutamate(1−) |
| THNSL1 | 185875 | threonine |
| THNSL2 | 144115 | threonine |
| TKT | 163931 | tryptophan |
| TKTL1 | 007350 | tryptophan |
| TKTL2 | 151005 | tryptophan |
| UMPS | 114491 | uracil |
| UROD | 126088 | heme |
| UROS | 188690 | heme |
| USF1 | 158773 | glutamate(1−) |
| USF2 | 105698 | glutamate(1−) |
| VPS33A | 139719 | methionine |
| VPS33B | 184056 | methionine |
| VPS36 | 136100 | ethanolamine |
| VPS4A | 132612 | ethanolamine |
| VPS4B | 119541 | ethanolamine |

CCBL1 may also be referred to as KYAT1. CCBL2 may also be referred to as KYAT3. DHFRL1 may also be referred to as DHFR2. PYCRL may also be referred to as PYCR3. HRSP12 may also be referred to as RIDA.

The auxotrophic factor may be one or two or more nutrients, enzymes, altered pH, altered temperature, non-organic molecules, non-essential amino acids, or altered concentrations of a moiety (compared to normal physiologic concentrations), or combinations thereof. All references to auxotrophic factor herein contemplate administration of multiple factors. Any factor is suitable as long as it is not toxic to the subject and is not bioavailable or present in a sufficient concentration in an untreated subject to sustain growth and reproduction of the modified host cell.

For example, the auxotrophic factor may be a nutrient that is a substance required for proliferation or that functions as a cofactor in metabolism of the modified host cell. Various auxotrophic factors are disclosed in Table 1. In certain embodiments, the auxotrophic factor is selected from biotin, alanine, aspartate, asparagine, glutamate, serine, uracil, valine and cholesterol. Biotin, also known as vitamin B7, is necessary for cell growth. In some instances, valine is needed for the proliferation and maintenance of hematopoietic stem cells. In some instances, the compositions disclosed herein are used to express the enzymes in HSCs that relieve the need for valine supplementation and thereby give those cells a selective advantage when valine is removed from the diet compared to the unmodified cells.

B. Transgene

Therapeutic entities encoded by the genome of the modified host cell may cause therapeutic effects, such as molecule trafficking, inducing cell death, recruitment of additional cells, or cell growth. In some embodiments, the therapeutic effect is expression of a therapeutic protein. In some embodiments, the therapeutic effect is induced cell death, including cell death of a tumor cell.

C. Control of Transgene Expression

In some instances, the transgene is optionally linked to one or more expression control sequences, including the gene's endogenous promoter, or heterologous constitutive or inducible promoters, enhancers, tissue-specific promoters, or post-transcriptional regulatory sequences. For example, one can use tissue-specific promoters (transcriptional targeting) to drive transgene expression or one can include regulatory sequences (microRNA (miRNA) target sites) in the RNA to avoid expression in certain tissues (post-transcriptional targeting). In some instances, the expression control sequence functions to express the therapeutic transgene following the same expression pattern as in normal individuals (physiological expression) (See Toscano et al., Gene Therapy (2011) 18, 117-127 (2011), incorporated herein by reference in its entirety for its references to promoters and regulatory sequences).

Constitutive mammalian promoters include, but are not limited to, the promoters for the following genes: hypoxanthine phosphoribosyl transferase (HPTR), adenosine deaminase, pyruvate kinase, α-actin promoter and other constitutive promoters. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Commonly used promoters including the CMV (cytomegalovirus) promoter/enhancer, EF1a (elongation factor 1a), SV40 (simian virus 40), chicken β-actin and CAG (CMV, chicken β-actin, rabbit β-globin), Ubiquitin C and PGK, all of which provide constitutively active, high-level gene expression in most cell types. Other constitutive promoters are known to those of ordinary skill in the art.

Inducible promoters are activated in the presence of an inducing agent. For example, the metallothionein promoter is activated to increase transcription and translation in the presence of certain metal ions. Other inducible promoters include alcohol-regulated, tetracycline-regulated, steroid-regulated, metal-regulated, nutrient-regulated promoters, and temperature-regulated promoters.

For liver-specific targeting: Natural and chimeric promoters and enhancers have been incorporated into viral and non-viral vectors to target expression of factor VIIa, factor VIII or factor IX to hepatocytes. Promoter regions from liver-specific genes such as albumin and human al antitrypsin (hAAT) are good examples of natural promoters. Alternatively, chimeric promoters have been developed to increase specificity and/or vectors efficiency. Good examples are the (ApoE)4/hAAT chimeric promoter/enhancer, harboring four copies of a liver-specific ApoE/hAAT enhancer/promoter combination and the DC172 chimeric promoter, consisting in one copy the hAAT promoter and two copies of the α(1)-microglobulin enhancer.

For muscle-specific targeting: Natural (creatine kinase promoter-MCK, desmin) and synthetic (α-myosin heavy chain enhancer-/MCK enhancer-promoter (MHCK7)) promoters have been included in viral and non-viral vectors to achieve efficient and specific muscle expression.

For endothelium-specific targeting, both natural (vWF, FLT-1 and ICAM-2) and synthetic promoters have been used to drive endothelium-specific expression.

For myeloid cell targeting, a synthetic chimeric promoter that contains binding sites for myeloid transcription factors CAAT box enhancer-binding family proteins (C/EBPs) and PU.1, which are highly expressed during granulocytic differentiation, has been reported to direct transgene expression primarily in myeloid cells (See, Santilli et al., Mol Ther. 2011 January; 19(1):122-32, which is hereby incorporated by reference in its entirety. CD68 may also be used for myeloid targeting.

Examples of tissue-specific vectors for gene therapy of genetic diseases are shown in Table 2.

TABLE 2
Tissue-specific vectors

| Promoter | Vector type | Target cell/tissue |
|---|---|---|
| WAS proximal promoter | HIV-1-based vectors | Hematopoietic cells |
| CD4 mini-promoter/enhancer | MLV-based vectors | T cells |
| CD2 locus control region | MLV based and HIV-1-based | T cells |
| | vectors | |
| CD4 minimal promoter and proximal enhancer and silencer | HIV-1-based vectors | T cells |
| CD4 mini-promoter/enhancer | HIV-1-based vectors | T cells |
| GATA-1 enhancer HS2 within the LTR | SFCM retroviral vector | Erythroid linage |
| Ankyrin-1 and α-spectrin promoters combined or not with HS- | HIV-1-based vectors | Erythroid linage |
| 40, GATA-1, ARE and intron 8 enhancers | | |
| Ankyrin-1 promoter/13-globin HS-40 enhancer | HIV-1-based vectors | Erythroid linage |
| GATA-1 enhancer HS1 to HS2 within the retroviral LTR | SFCM retroviral vector | Erythroid linage |
| Hybrid cytomegalovirus (CMV) enhancer/β-actin promoter | Sleeping Beauty transposon | Erythroid linage |
| MCH II-specific HLA-DR promoter | HIV-1-based vectors | APCs |
| Fascin promoter (pFascin) | Plasmid | APCs |
| Dectin-2 gene promoter | HIV-1-based vectors | APCs |
| 5′ untranslated region from the DC-STAMP | HIV-1-based vectors | APCs |
| Heavy chain intronic enhancer (Eμ) and matrix attachment | HIV-1-based vectors | B cells |
| regions | | |
| CD19 promoter | MLV based vectors | B cells |
| Hybrid immunoglobulin promoter (Igk promoter, intronic | HIV-1-based vectors | B cells |
| Enhancer and 3′ enhancer from Ig genes) | | |
| CD68L promoter and first intron | MLV-based vectors | Megakaryocytes |
| Glycoprotein Ibα promoter | HIV-1-based vectors | Megakaryocytes |
| Apolipoprotein E (Apo E) enhancer/alpha1-antitrypsin (hAAT) | MLV based vectors | Hepatocytes |
| promoter (ApoE/hAAT) | | |
| HAAT promoter/Apo E locus control region | Plasmid | Hepatocytes |
| Albumin promoter | HIV-1-based vectors | Hepatocytes |
| HAAT promoter/four copies of the Apo E enhancer | AAV-2-based vectors | Hepatocytes |
| Albumin and hAAT promoters/α1-microglobulin and | Plasmid | Hepatocytes |
| prothrombin enhancers | | |
| HAAT promoter/Apo E locus control region | AAV8 | Hepatocytes |
| hAAT promoter/four copies of the Apo E enhancer | AAV2/8 | Hepatocytes |
| TBG promoter (thyroid hormone-binding globulin promoter | AAV | Hepatocytes |
| and α1-microglobulin/bikunin enhancer) | | |
| DC172 promoter (α1-antitrypsin promoter and α1- | Adenovirus, plasmid | Hepatocytes |
| microglobulin enhancer) | | |
| LCAT, kLSP-IVS, ApoE/hAAT and liver-fatty acid-binding | AAV1, AAV2, AAV6, AAV8 | Hepatocytes |
| protein promoters | | |
| RU486-responsive promoter | Adenovirus | Hepatocytes |
| Creatine kinase promoter | Adenovirus | Muscle |
| Creatine kinase promoter | AAV6 | Muscle |
| Synthetic muscle-specific promoter C5-12 | AV-1 | Muscle |
| Creatine kinase promoter | AAV2/6 | Muscle |
| Hybrid enhancer/promoter regions of α-myosin and creatine | AAV6 | Muscle |
| kinase (MHCK7) | | |
| Hybrid enhancer/promoter regions of α-myosin and creatine | AAV2/8 | Muscle |
| kinase | | |
| Synthetic muscle-specific promoter C5-12 | HIV-1-based vectors | Muscle |
| Cardiac troponin-I proximal promoter | HIV-1-based vectors | Cardiomyocytes |
| E-selectin and KDR promoters | MLV-based vectors | Endothelial cell |
| Prepro-endothelin-1 promoter | MLV-based vectors | Endothelial cell |
| KDR promoter/hypoxia-responsive element | MLV-based vectors | Endothelial cell |
| Flt-1 promoter | Adenovirus | Endothelial cell |
| Flt-1 promoter | Adenovirus | Endothelial cell |
| ICAM-2 promoter | Plasmid | Endothelial cell |
| Synthetic endothelial promoter | HIV-1-based vectors | Endothelial cell |
| Endothelin-1 gene promoter | Sleeping Beauty transposon | Endothelial cell |
| Amylase promoter | Adenovirus | Pancreas |
| Insulin and human pdx-1 promoters | Adenovirus | Pancreas |
| TRE-regulated insulin promoter | Plasmid | Pancreas |
| Enolase promoter | Herpesvirus | Neurons |
| Enolase promoter | Adenoviruses | Neurons |
| TRE-regulated synapsin promoter | Adenoviruses | Neurons |
| Synapsin 1 promoter | Adenoviruses | Neurons |
| PDGF-β promoter/CMV enhancer | Plasmid | Neurons |
| PDGF-β synapsin, tubulin-α and ca2+/calmodulin-PK2 | HIV-1-based vectors | Neurons |
| promoters combined with CMV enhancer | | |
| Phosphate-activated glutaminase and vesicular glutamate | Herpesvirus | Glutamatergic |
| transporter-1 promoters | | neurons |
| Glutamic acid decarboxylase-67 promoter | Herpesvirus | GABAergic neuron |
| Tyrosine hydroxylase promoter | Herpesvirus | Catecholaminergic |
| | | neurons |
| Neurofilament heavy gene promoter | Herpesvirus | Neurons |
| Human red opsin promoter | Recombinant AAV | Cone cells |
| Keratin-18 promoter | Adenovirus | Epithelial cells |
| keratin-14 (K14) promoter | Lentiviral vectors | Epithelial cells |
| Keratin-5 promoter | HIV-1-based vectors | Epithelial cells |

Examples of physiologically regulated vectors for gene therapy of genetic diseases are shown in Table 3.

TABLE 3
Physiologically regulated vectors

| Promoter | Vector type | Target cell/tissue |
|---|---|---|
| WAS proximal promoter (1600 bp) | HIV-1-based vectors | Hematopoietic cells |
| WAS proximal promoter (500 bp) | HIV-1-based vectors | Hematopoietic cells |
| WAS proximal promoter (170 bp) | HIV-1-based vectors | Hematopoietic cells |
| WAS proximal promoter (500 bp)/WAS | HIV-1-based vectors | Hematopoietic cells |
| alternative promoter (386 bp) | | |
| CD40L promoter and regulatory sequences | Human artificial chromosome (HAC) | Activated T cells |
| CD40L promoter | HIV-1-based vectors | Activated T cells |
| β-Globin promoter/LCR | HIV-1-based vectors | Erythroid linage |
| β-Globin and θ-globin promoters combined or | HIV-1-based vectors | Erythroid linage |
| not with HS-40, GATA-1, ARE, and intron 8 | | |
| enhancers | | |
| β-Globin, LCR HS4, HS3, HS2 and a truncated | HIV-1-based vectors | Erythroid linage |
| β-globin intron 2 | | |
| β-Globin promoter/LCR/cHS4 | HIV-1-based vectors | Erythroid linage |
| HSFE/LCR/β-globin promoter | MSCV retroviral vector | Erythroid linage |
| Integrin αIIb promoter (nucleotides −889 to +35) | MLV-based vectors | Megakaryocytes |
| Dystrophin promoter and regulatory sequences | HAC | Muscle |
| Endoglin promoter | Plasmid | Endothelial cells |
| RPE65 promoter | AAV2/4 | Retinal pigmented epithelium |
| TRE-regulated synapsin promoter | Adenoviruses | Neurons |

Tissue-specific and/or physiologically regulated expression can also be pursued by modifying mRNA stability and/or translation efficiency (post-transcriptional targeting) of the transgenes. Alternatively, the incorporation of miRNA target recognition sites (miRTs) into the expressed mRNA has been used to recruit the endogenous host cell machinery to block transgene expression (detargeting) in specific tissues or cell types. miRNAs are noncoding RNAs, approximately 22 nucleotides, that are fully or partially complementary to the 3′ UTR region of particular mRNA, referred to as miRTs. Binding of a miRNA to its particular miRTs promotes translational attenuation/inactivation and/or degradation. Regulation of expression through miRNAs is described in Geisler and Fechner, World J Exp Med. 2016 May 20, 6(2): 37-54; Brown and Naldini, Nat Rev Genet. 2009 Aug. 10(8):578-85; Gentner and Naldini, Tissue Antigens. 2012 November, 80(5):393-403; each of which is hereby incorporated by reference in its entirety. Engineering miRTs-vector recognized by a specific miRNA cell type has been shown to be an effective way for knocking down the expression of a therapeutic gene in undesired cell types (See, Toscano et al., supra., which is hereby incorporated by reference in its entirety).

D. Pharmaceutical Compositions

Disclosed herein, in some embodiments, are methods, compositions and kits for use of the modified cells, including pharmaceutical compositions, therapeutic methods, and methods of administration of auxotrophic factors to control—increase, decrease or cease—the growth and reproduction of the modified cells and to control the expression of the therapeutic factor by the transgene.

The modified mammalian host cell may be administered to the subject separately from the auxotrophic factor or in combination with the auxotrophic factor. Although the descriptions of pharmaceutical compositions provided herein are principally directed to pharmaceutical compositions which are suitable for administration to humans, it will be understood by the skilled artisan that such compositions are generally suitable for administration to any animals.

Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys. In some embodiments, compositions are administered to humans, human patients, or subjects.

In some instances, the pharmaceutical compositions described herein is used in a method of treating a disease, a disorder, or a condition in a subject, the method including: (i) generating a cell line which is auxotrophic for a nutrient, an enzyme, an altered pH, an altered temperature, an altered concentration of a moiety, and/or a niche environment, such that the nutrient, enzyme, altered pH, altered temperature, and niche environment is not present in the subject; (ii) contacting the subject with the resulting auxotrophic cell line of step (i); (iii) contacting the subject of (ii) with the auxotrophic factor which is selected from the nutrient, enzyme, moiety that alters pH and/or temperature, and a cellular niche environment in the subject, such that the auxotrophic factor activates the auxotrophic system or element resulting in the growth of the cell line and/or the expression of one or more therapeutic entities for the subject.

The pharmaceutical compositions of the disclosure herein may also be used in a method of treating a disease, a disorder, or a condition in a subject, comprising (a) administering to the subject a modified host cell according to the disclosure herein, and (b) administering the auxotrophic factor to the subject in an amount sufficient to promote growth of the modified host cell.

Compositions comprising a nutrient auxotrophic factor may also be used for administration to a human comprising a modified host cell of the disclosure herein.

### V. Formulations

A. Cellular Engineering Formulations

The modified host cell is genetically engineered to insert the transgene encoding the therapeutic factor into the auxotrophy-inducing locus. Delivery of Cas9 protein/gRNA ribonucleoprotein complexes (Cas9 RNPs) targeting the desired locus may be performed by liposome-mediated transfection, electroporation, or nuclear localization. In some embodiments, the modified host cell is in contact with a medium containing serum following electroporation. In some embodiments, the modified host cell is in contact with a medium containing reduced serum or containing no serum following electroporation.

B. Therapeutic Formulations

The modified host cell or auxotrophic factor of the disclosure herein may be formulated using one or more excipients to: (1) increase stability; (2) alter the biodistribution (e.g., target the cell line to specific tissues or cell types); (3) alter the release profile of an encoded therapeutic factor; and/or (4) improve uptake of the auxotrophic factor.

Formulations of the present disclosure can include, without limitation, saline, liposomes, lipid nanoparticles, polymers, peptides, proteins, and combinations thereof.

Formulations of the pharmaceutical compositions described herein may be prepared by any method known or hereafter developed in the art of pharmacology. As used herein the term “pharmaceutical composition” refers to compositions including at least one active ingredient and optionally one or more pharmaceutically acceptable excipients. Pharmaceutical compositions of the present disclosure may be sterile.

In general, such preparatory methods include the step of associating the active ingredient with an excipient and/or one or more other accessory ingredients. As used herein, the phrase “active ingredient” generally refers to either (a) a modified host cell or donor template including a transgene capable of expressing a therapeutic factor inserted into an auxotrophy-inducing locus, or (b) the corresponding auxotrophic factor, or (c) the nuclease system for targeting cleavage within the auxotrophy-inducing locus.

Formulations of the modified host cell or the auxotrophic factor and pharmaceutical compositions described herein may be prepared by a variety of methods known in the art.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition including a predetermined amount of the active ingredient.

Relative amounts of the active ingredient (e.g. the modified host cell or auxotrophic factor), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

C. Excipients and Diluents

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

D. Inactive Ingredients

In some embodiments, formulations may include at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the U.S. Food and Drug Administration (FDA).

E. Pharmaceutically Acceptable Salts

The auxotrophic factor may be administered as a pharmaceutically acceptable salt thereof. As used herein, “pharmaceutically acceptable salts” refers to derivatives of the disclosed compounds such that the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecyl sulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

### VI. Dosing and Administration

The modified host cells or auxotrophic factors of the present disclosure included in the pharmaceutical compositions described above may be administered by any delivery route, systemic delivery or local delivery, which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal, (through the eye), intracavernous injection (into a pathologic cavity), intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

A. Parenteral and Injectable Administration

In some embodiments, the modified host cells may be administered parenterally.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

B. Depot Administration

As described herein, in some embodiments, pharmaceutical compositions including the modified host cell of the present disclosure are formulated in depots for extended release. Generally, specific organs or tissues (“target tissues”) are targeted for administration. In some embodiments, localized release is affected via utilization of a biocompatible device. For example, the biocompatible device may restrict diffusion of the cell line in the subject.

In some aspects of the disclosure herein, pharmaceutical compositions including the modified host cell of the present disclosure are spatially retained within or proximal to target tissues. Provided are methods of providing pharmaceutical compositions including the modified host cell or the auxotrophic factor, to target tissues of mammalian subjects by contacting target tissues (which include one or more target cells) with pharmaceutical compositions including the modified host cell or the auxotrophic factor, under conditions such that they are substantially retained in target tissues, meaning that at least 10, 20, 30, 40, 50, 60, 70, 80, 85, 90, 95, 96, 97, 98, 99, 99.9, 99.99, or greater than 99.99% of the composition is retained in the target tissues. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of pharmaceutical compositions including the modified host cell or the auxotrophic factor administered to subjects are present at a period of time following administration.

Certain aspects of the present disclosure are directed to methods of providing pharmaceutical compositions including the modified host cell or the auxotrophic factor of the present disclosure to target tissues of mammalian subjects, by contacting target tissues with pharmaceutical compositions including the modified host cell under conditions such that they are substantially retained in such target tissues. Pharmaceutical compositions including the modified host cell include enough active ingredient such that the effect of interest is produced in at least one target cell. In some embodiments, pharmaceutical compositions including the modified host cell generally include one or more cell penetration agents, although “naked” formulations (such as without cell penetration agents or other agents) are also contemplated, with or without pharmaceutically acceptable excipients.

C. Therapeutic Methods

The present disclosure additionally provides a method of delivering to a subject, including a mammalian subject, any of the above-described modified host cells or auxotrophic factors including as part of a pharmaceutical composition or formulation.

D. Dose and Regimen

The present disclosure provides methods of administering modified host cells or auxotrophic factors in accordance with the disclosure to a subject in need thereof. The pharmaceutical compositions including the modified host cell or the auxotrophic factor, and compositions of the present disclosure may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, route of administration, and rate of excretion of the auxotrophic factor; the duration of the treatment; drugs used in combination or coincidental with the specific modified host cell or auxotrophic factor employed; and like factors well known in the medical arts.

In certain embodiments, modified host cell or the auxotrophic factor pharmaceutical compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

In certain embodiments, modified host cell or auxotrophic factor pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site. As non-limiting examples, the modified host cell or auxotrophic factor may be administered at 50 μl/site and/or 150 μl/site.

The desired dosage of the modified host cell or auxotrophic factor of the present disclosure may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

The desired dosage of the modified host cells of the present disclosure may be administered one time or multiple times. The auxotrophic factor is administered regularly with a set frequency over a period of time, or continuously as a “continuous flow”. A total daily dose, an amount given or prescribed in 24-hour period, may be administered by any of these methods, or as a combination of these methods.

In some embodiments, delivery of the modified host cell or auxotrophic factor of the present disclosure to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

The modified host cells may be used in combination with one or more other therapeutic, prophylactic, research or diagnostic agents, or medical procedures, either sequentially or concurrently. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

For example, the modified host cell or auxotrophic factor is administered as a biocompatible device that restricts diffusion in the subject to increase bioavailability in the area targeted for treatment. The modified host cell or auxotrophic factor may also be administered by local delivery.

The disclosure herein contemplates methods of expressing a therapeutic factor in a subject comprising (a) administering said modified cells, (b) optionally administering a conditioning regime to permit modified cells to engraft, and (c) administering said auxotrophic factor.

The term “conditioning regime” refers to a course of therapy that a patient undergoes before stem cell transplantation. For example, before hematopoietic stem cell transplantation, a patient may undergo myeloablative therapy, non-myeloablative therapy or reduced intensity conditioning to prevent rejection of the stem cell transplant even if the stem cell originated from the same patient. The conditioning regime may involve administration of cytotoxic agents. The conditioning regime may also include immunosuppression, antibodies, and irradiation. Other possible conditioning regiments include antibody mediated conditioning (see e.g., Czechowicz et al., 318(5854) Science 1296-9 (2007); Palchaudari et al., 34(7) Nature Biotechnology 738-745 (2016); Chhabra et al., 10:8(351) Science Translational Medicine 351ra105 (2016)) and CAR-T mediated conditioning (see, e.g., Arai et al., 26(5) Molecular Therapy 1181-1197 (2018); each of which is hereby incorporated by reference in its entirety). Conditioning needs to be used create space in the brain for microglia derived from engineered HSCs to migrate into to deliver the protein of interest (recent gene therapy trials for ALD and MLD). The conditioning regimen is also designed to create niche “space” to allow the transplanted cells to have a place in the body to engraft and proliferate. In hematopoietic stem cell transplantation, for example, the conditioning regimen creates niche space in the bone marrow for the transplanted hematopoietic stem cells to engraft into. Without a conditioning regimen the transplanted hematopoietic stem cells cannot engraft. In some embodiments, the cell lines are T cells that are genetically engineered to be auxotrophic. Engineered auxotrophic T cells may be used as CAR T cells to act as a living drug and administered to a patient along with an auxotrophic factor to condition the patient for a hematopoietic stem cell transplant. Prior to the delivery of the donor hematopoietic stem cells, the auxotrophic factor may be removed, which results in the elimination of the engineered auxotrophic T cells. In some embodiments, the cell lines are allogenic T cells that are genetically engineered to be auxotrophic. Engineered auxotrophic allogenic T cells may be administered to a patient along with an auxotrophic factor to provide a therapeutic effect. Upon the patient developing graft-versus-host disease (GvHD), the auxotrophic factor may be removed, which results in the elimination of the engineered auxotrophic allogenic T cells which have become alloreactive.

In some embodiments, administration of said auxotrophic factor is continued regularly for a period of time sufficient to express the therapeutic factor, and preferably for a period of time sufficient for the therapeutic factor to exert a therapeutic effect. In some embodiments, administration of said auxotrophic factor is decreased to decrease expression of the therapeutic factor. In some embodiments, administration of said auxotrophic factor is increased to increase expression of the therapeutic factor. In some embodiments, administration of said auxotrophic factor is discontinued to create conditions that result in growth inhibition or death of the modified cells. In some embodiments, administration of said auxotrophic factor is temporarily interrupted to create conditions that result in growth inhibition of the modified cells.

The disclosure herein also contemplates a method of treating a subject with a disease, a disorder, or a condition comprising administering to the subject (a) said modified mammalian host cells and (b) said auxotrophic factor in an amount sufficient to produce expression of a therapeutic amount of the therapeutic factor.

Use of a modified mammalian host cell according to the present disclosure for treatment of a disease, disorder or condition is also encompassed by the disclosure.

Certain embodiments provide the disease, the disorder, or the condition as selected from the group consisting of cancer, Parkinson's disease, graft versus host disease (GvHD), autoimmune conditions, hyperproliferative disorder or condition, malignant transformation, liver conditions, genetic conditions including inherited genetic defects, juvenile onset diabetes mellitus and ocular compartment conditions.

In certain embodiments, the disease, the disorder, or the condition affects at least one system of the body selected from the group consisting of muscular, skeletal, circulatory, nervous, lymphatic, respiratory endocrine, digestive, excretory, and reproductive systems. Conditions that affect more than one cell type in the subject may be treated with more than one modified host cell with each cell line activated by a different auxotrophic factor. In some cases, a subject may be administered more than one auxotrophic factor.

Certain embodiments provide the cell line as regenerative. In an aspect of the present disclosure, the subject may be contacted with more than one modified host cell and/or with one or more auxotrophic factor. Certain embodiments provide localized release of the auxotrophic factor, e.g. nutrient or the enzyme. Alternative embodiments provide systemic delivery. For example, localized release is affected via utilization of a biocompatible device. In an aspect of the present disclosure, the biocompatible device may restrict diffusion of the cell line in the subject. Certain embodiments of the method provide removing the auxotrophic factor to deplete therapeutic effects of the modified host cell in the subject or to induce cell death in the modified host cell. Certain embodiments of the method provide the therapeutic effects as including at least one selected from the group consisting of: molecule trafficking, inducing cell death, cell death, and recruiting of additional cells. Certain embodiments of the method provide that the unmodified host cells are derived from the same subject prior to treatment of the subject with the modified host cells.

The present disclosure contemplates kits comprising such compositions or components of such compositions, optionally with a container or vial.

### VII. Definitions

The term “about” in relation to a numerical value x means, for example, x±10%.

The term “active ingredient” generally refers to the ingredient in a composition that is involved in exerting a therapeutic effect. As used herein, it generally refers to (a) the modified host cell or donor template including a transgene as described herein, (b) the corresponding auxotrophic factor as described herein, or (c) the nuclease system for targeting cleavage within the auxotrophy-inducing locus.

The term “altered concentration” as used herein, refers to an increase in concentration of an auxotrophic factor compared to the concentration of the auxotrophic factor in the subject prior to administration of the pharmaceutical compositions described herein.

The term “altered pH” as used herein, refers to a change in pH induced in a subject compared to the pH in the subject prior to administration of the pharmaceutical composition described herein.

The term “altered temperature” as used herein refers to a change in temperature induced in a subject compared to the temperature in the subject prior to administration of the pharmaceutical composition as described herein.

The term “auxotrophy” or “auxotrophic” as used herein, refers to a condition of a cell that requires the exogenous administration of an auxotrophic factor to sustain growth and reproduction of the cell.

The term “auxotrophy-inducing locus” as used herein refers to a region of a chromosome in a cell that, when disrupted, causes the cell to be auxotrophic. For example, a cell can be rendered auxotrophic by disrupting a gene encoding an enzyme involved in synthesis, recycling or salvage of an auxotrophic factor (either directly or upstream through synthesizing intermediates used to make the auxotrophic factor), or by disrupting an expression control sequence that regulates the gene's expression.

The term “bioavailability” as used herein, refers to systemic availability of a given amount of the modified host cell or auxotrophic factor administered to a subject.

The term “Cas9” as used herein, refers to CRISPR-associated protein 9, which is an endonuclease for use in genome editing.

The term “comprising” means “including” as well as “consisting” e.g. a composition “comprising” X may consist exclusively of X or may include something additional e.g. X+Y.

The term “conditioning regime” refers to a course of therapy that a patient undergoes before stem cell transplantation.

The term “continuous flow” as used herein, refers to a dose of therapeutic administered continuously for a period of time in a single route/single point of contact, i.e., continuous administration event.

The term “CRISPR” as used herein, refers to clustered regularly interspaced short palindromic repeats of DNA that deploy an enzyme that cuts the RNA nucleotides of an invading cell.

The term “CRISPR/Cas9 nuclease system” as used herein, refers to a genetic engineering tool that includes a guide RNA (gRNA) sequence with a binding site for Cas9 and a targeting sequence specific for the site to be cleaved in the target DNA. The Cas9 binds the gRNA to form a ribonucleoprotein complex that binds and cleaves the target site.

The term “expanding” when used in the context of cells refers to increasing the number of cells through generation of progeny.

The term “expression control sequence” refers to a nucleotide sequence capable of regulating or controlling expression of a nucleotide sequence of interest. Examples include a promoter, enhancer, transcription factor binding site, miRNA binding site.

The term “homologous recombination” (HR) refers to insertion of a nucleotide sequence during repair of breaks in DNA via homology-directed repair mechanisms. This process uses a “donor” molecule or “donor template” with homology to nucleotide sequence in the region of the break as a template for repairing the break. The inserted nucleotide sequence can be a single base change in the genome or the insertion of large sequence of DNA.

The term “homologous” or “homology,” when used in the context of two or more nucleotide sequences, refers to a degree of base pairing or hybridization that is sufficient to specifically bind the two nucleotide sequences together in a cell under physiologic conditions.

Homology can also be described by calculating the percentage of nucleotides that would undergo Watson-Crick base pairing with the complementary sequence, e.g. at least 70% identity, preferably at least 75%, 80%, 85%, 90%, 91%, 92%, 93%, 94%, 95%, 96%, 97%, 98%, 99%, or higher identity over a specified number of bases. With respect to donor templates, for example, the homology may be over 200-400 bases. With respect to guide sequences, for example, the homology may be over 15-20 bases.

The term “operatively linked” refers to functional linkage between a nucleic acid expression control sequence (such as a promoter, enhancer, signal sequence, or array of transcription factor binding sites) and a second nucleic acid sequence, wherein the expression control sequence affects transcription and/or translation of the second nucleic acid sequence.

The term “pharmaceutical composition” as used herein, refers to a composition including at least one active ingredient and optionally one or more pharmaceutically acceptable excipients.

The term “pharmaceutically acceptable salt” as used herein, refers to derivatives of the disclosed compounds such that the parent compound is modified by converting an existing acid or base moiety to its salt form (e.g., by reacting the free base group with a suitable organic acid). All references herein to compounds or components include the pharmaceutically acceptable salt thereof.

The term “regenerative” as used herein, refers to renewal or restoration of an organ or system of the subject.

The term “therapeutic factor” refers to a product encoded by the inserted transgene that treats and/or alleviates symptoms of the disease, disorder, or condition of the subject.

The term “therapeutic amount” refers to an amount of therapeutic factor sufficient to exert a “therapeutic effect”, which means an alleviation or amelioration of symptoms of the disease, disorder or condition.

The term “unit dose” as used herein, refers to a discrete amount of the pharmaceutical composition including a predetermined amount of the active ingredient.

### EXAMPLES

### Example 1. General T Cell Culture Methods

K562 cells (acquired from ATCC) and Nalm6 cells (kindly provided by C. Mackall) were cultured in RPMI 1640 (HyClone) supplemented with 10% bovine growth serum, 2 mM L-glutamine and 100 U/ml Penicillin and 100 U/ml Streptomycin. T cells were either used fresh after isolation from buffy coats obtained from healthy donors. T cells were isolated through a Ficoll density gradient centrifugation followed by magnetic enrichment using the Pan T Cell Isolation Kit (Miltenyi Biotec).

Cells were cryopreserved in BAMBANKER™ medium. After thawing cells were cultured at 37° C., 5% CO₂ in X-Vivo 15 (Lonza) supplemented with or without 5% human serum (Sigma-Aldrich) and 100 human recombinant IL-2 (Peprotech) and 10 ng/ml human recombinant IL-7 (BD Biosciences). UMP or Uridine was added at 250 μg/ml. 5-FOA was added at 100 μg/ml to 1 mg/ml. During culture, medium was refreshed every 2 days.

T cells were activated using immobilized Anti-CD3 (clone OKT3, Tonbo Biosciences) and soluble anti-CD28 (clone CD28.2, Tonbo Biosciences) for three days before electroporation.

1.4 million activated T cells were resuspended in electroporation solution, mixed with the pre-complexed RNP, and electroporated using a 4D-NUCLEOFECTOR™ system (Lonza) using program EO-115. The RNP consisted of Cas9 protein (Alt-R® CRISPR/Cas9 system based on S. pyogenes, IDT) at 300 μg/ml and sgRNA using a sgRNA:Cas9 molar ratio of 2.5.

Genomic DNA was harvested using QUICKEXTRACT™ DNA Extraction Kit (Epicentre). Cells were counted on an automated cell counter using Trypan blue staining or on a CytoFLEX flow cytometer (Beckman Coulter) with automatic plate reader using COUNTBRIGHT™ beads (ThermoFisher) as a reference for normalizing the values. Alternatively, cells were analyzed after staining with fluorochrome-labelled antibodies (Biolegend) on an ACCURI™ C6 flow cytometer (BD Biosciences), which also measures volumes, or a FACS ARIA™ II SORP cell sorter (BD Biosciences). Data was analyzed using Excel (Microsoft) and FlowJo software (Tree Star).

Sanger sequencing of the UMPS locus was performed using UMPS-O-1 and UMPS-O-2, with the region amplified using PHUSION™ Hot Start Flex 2x Master Mix (New England Biolabs, Inc.). Sanger sequencing traces were analyzed by TIDE analysis (see, Brinkman et al, 2014, Nucleic Acids Res. 42(22):e168), which is hereby incorporated by reference in its entirety) to identify insertions and deletions (InDels) after editing. InDel quantification was performed on the sequences using the TIDE online tool (www.deskgen.com/landing/tide.html) (See, M. Sadelain, N. Engl. J. Med. 365, 1735-7 (2011), which is hereby incorporated by reference in its entirety.

gRNA sequences (including protospacer adjacent motifs, also referred to as PAMs):

UMPS-7

(SEQ ID NO: 1)
GCC CCG CAG AUC GAU GUA GAG UUU UAG AGC UAG
AAA UAG CAA GUU AAA AUA AGG CUA GUC CGU UAU
CAA CUU GAA AAA GUG GCA CCG AGU CGG UGC UUU
U

Sequencing oligonucleotides for UMPS locus TIDE analysis:

UMPS-O-1:
(SEQ ID NO: 2)
CCCGGGGAAACCCACGGGTGC
UMPS-O-2:
(SEQ ID NO: 3)
AGGGTCGGTCTGCCTGCTTGGCT

After the initial screening, sgRNA “UMPS-7,” which showed the highest frequency of InDels was chosen for further analysis,

### Example 2. UMPS Editing by Cas9-sgRNA Electroporation in Human T Cells

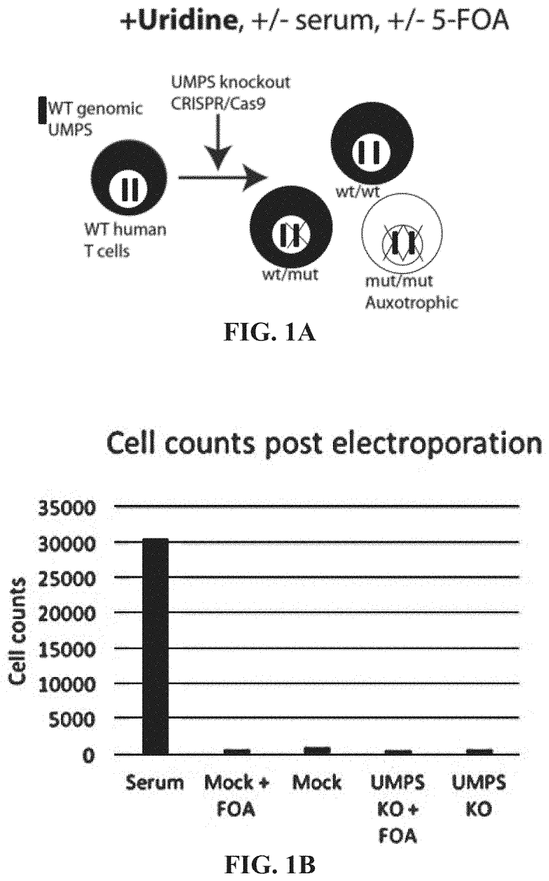

T Cells were thawed and cultured, followed by activation and subsequent electroporation with Cas9-UMPS-7 sgRNA RNP as described above. Following electroporation, cells were allowed to recover in medium with or without serum, 5-FOA or an exogenous uracil source (FIG. 1A). Cell survival following electroporation was markedly increased when serum was included in the media (FIG. 1B), and thus a four-day recovery period in medium with serum, uridine, and UMP was performed in all subsequent experiments. Cell counts post-electroporation are shown in Table 4.

TABLE 4
Cell counts

| Sample | Intact cells (absolute) |
|---|---|
| Serum | 30217 |
| Mock + FOA | 580 |
| Mock | 901 |
| UMPS KO + FOA | 395 |
| UMPS KO | 560 |

### Example 3. Growth of a Mixed UMPS Edited Population and Maintenance of UMPS Mutations

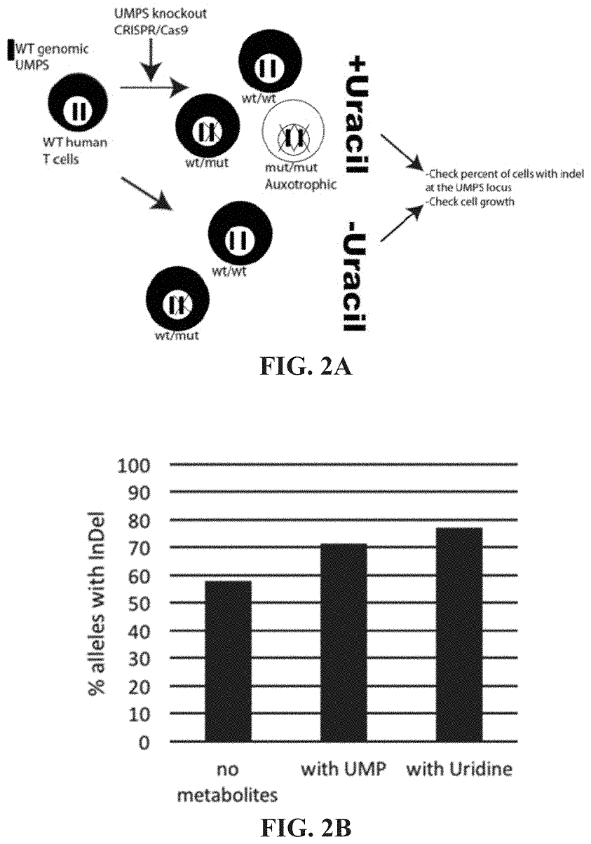

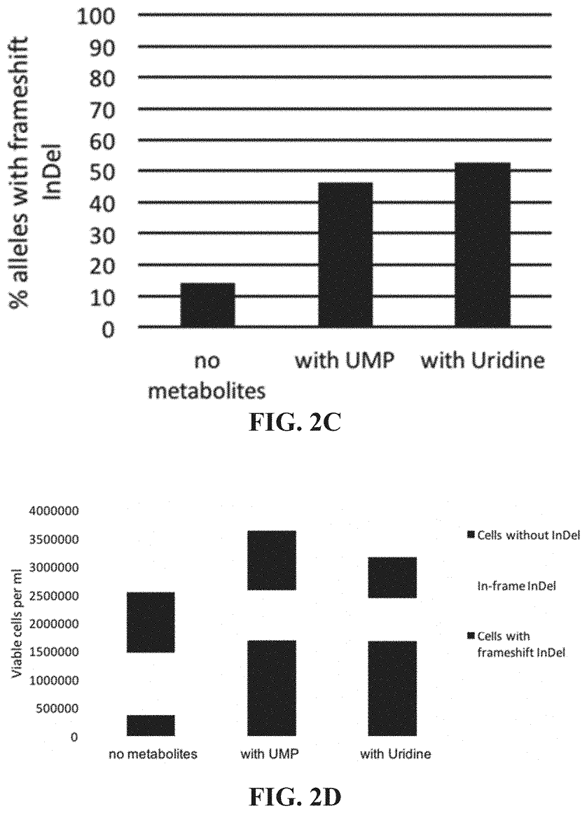

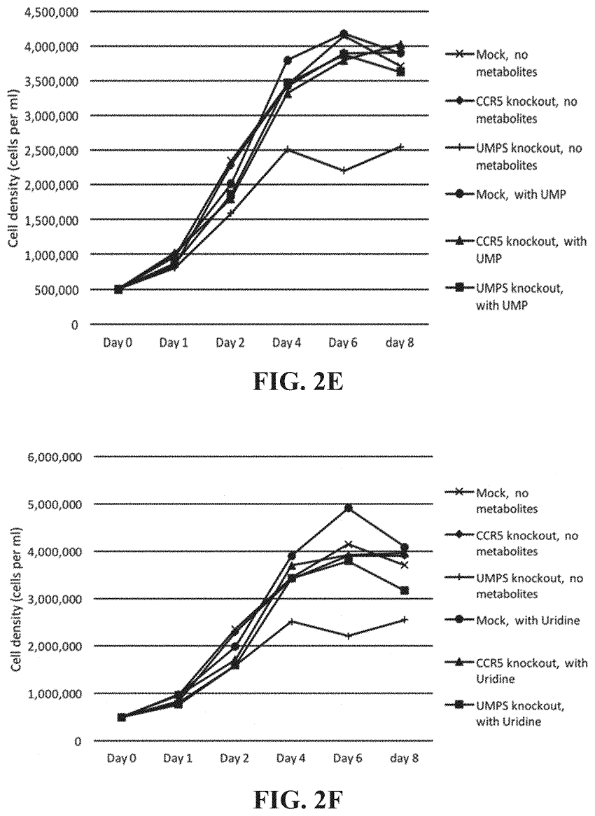

T Cells were electroporated and edited as in Example 2 and allowed to recover for a 4-day period in medium with serum, uridine, and UMP. On day 4, cells were shifted to UMP, uridine, or uracil source media. This experiment did not feature a selection step and thus the resulting population of cells was a heterogeneous mix of wild-type (WT), heterozygous mutant and homozygous mutant cells. The growth of homozygous UMPS mutant cells was observed to be dependent on an exogenous uracil source—as these should be auxotrophic (FIG. 2A). When UMPS is targeted, InDels were observed to be generated in about 50% of cells (as assayed by TIDE analysis (See, Brinkman et al, 2014, Nucleic Acids Res. 42(22):e168), which is hereby incorporated by reference in its entirety).

When the exogenous uracil source was removed, the InDel frequency in the population was reduced after three days of growth (Day 7=four days of recovery and three days in test media). This was consistent with the model showing that any homozygous auxotrophic UMPS mutant cells would be outcompeted in the population by non-auxotrophic heterozygous mutants and WT cells still present after editing—resulting in a reduced apparent InDel frequency (see, FIG. 2B). The percentages of alleles with InDels are shown in Table 5.

TABLE 5
Alleles with InDels

| Condition | Percent of alleles (without 5-FOA) |
|---|---|
| no metabolites | 57.9 |
| with UMP | 71.1 |
| with Uridine | 77.0 |

The optimal growth of the heterogeneous UMPS edited population was observed to be dependent on the presence of an exogenous source of uracil (FIG. 2C-FIG. 2F). The percent of alleles with a frameshift InDel is shown in FIG. 2C, and the values are shown in Table 6.

TABLE 6
Alleles with frameshift InDels

| | Percent of alleles |
|---|---|
| | (without 5-FOA) |
| no metabolites | 14.3 |
| with UMP | 46.1 |
| with Uridine | 52.5 |

FIG. 2D compares the predicted absolute numbers of cells at day 8 containing alleles identified by TIDE. The values are shown in Table 7.

TABLE 7
Predicted viable cell counts

| | Cells with | Cells with | Cells without |
|---|---|---|---|
| Condition | frameshift InDel | In-frame InDel | InDel |
| no metabolites | 365000 | 1110000 | 1073550 |
| with UMP | 1670000 | 908000 | 1049070 |
| with Uridine | 1660000 | 777000 | 729100 |

FIG. 2E shows the time course (eight days) of cell counts with/without UMP. The values are shown in Table 8.

TABLE 8
Cell density [cells per ml]

| Treatment | Metabolite | Day 0 | Day 1 | Day 2 | Day 4 | Day 6 | Day 8 |
|---|---|---|---|---|---|---|---|
| Mock | no metabolites | 5.00E+05 | 9.67E+05 | 2.35E+06 | 3.44E+06 | 4.15E+06 | 3.71E+06 |
| CCR5 knockout | no metabolites | 5.00E+05 | 8.35E+05 | 2.29E+06 | 3.42E+06 | 3.90E+06 | 3.91E+06 |
| UMPS knockout | no metabolites | 5.00E+05 | 8.08E+05 | 1.59E+06 | 2.51E+06 | 2.21E+06 | 2.55E+06 |
| Mock | with UMP | 5.00E+05 | 9.83E+05 | 2.01E+06 | 3.80E+06 | 4.18E+06 | 3.90E+06 |
| CCR5 knockout | with UMP | 5.00E+05 | 1.02E+06 | 1.80E+06 | 3.32E+06 | 3.80E+06 | 4.03E+06 |
| UMPS knockout | with UMP | 5.00E+05 | 8.74E+05 | 1.86E+06 | 3.47E+06 | 3.88E+06 | 3.63E+06 |

FIG. 2F shows the time course (eight days) of cell counts with/without uridine. The values are shown in Table 9.

TABLE 9
Cell density [cells per ml]

| Treatment | Metabolite | Day 0 | Day 1 | Day 2 | Day 4 | Day 6 | Day 8 |
|---|---|---|---|---|---|---|---|
| Mock | no metabolites | 5.00E+05 | 9.67E+05 | 2.35E+06 | 3.44E+06 | 4.15E+06 | 3.71E+06 |
| CCR5 knockout | no metabolites | 5.00E+05 | 8.35E+05 | 2.29E+06 | 3.42E+06 | 3.90E+06 | 3.91E+06 |
| UMPS knockout | no metabolites | 5.00E+05 | 8.08E+05 | 1.59E+06 | 2.51E+06 | 2.21E+06 | 2.55E+06 |
| Mock | with Uridine | 5.00E+05 | 9.78E+05 | 1.98E+06 | 3.90E+06 | 4.91E+06 | 4.09E+06 |
| CCR5 knockout | with Uridine | 5.00E+05 | 9.67E+05 | 1.71E+06 | 3.70E+06 | 3.92E+06 | 3.96E+06 |
| UMPS knockout | with Uridine | 5.00E+05 | 7.69E+05 | 1.59E+06 | 3.43E+06 | 3.79E+06 | 3.17E+06 |

UMP and uridine rescued the growth of an UMPS edited culture to the same level as mock edited cells. This rescue of growth is dependent on UMPS editing and is not seen in mock cells treated with an exogenous uracil source, indicating that edited UMPS makes human T cells specifically dependent on uracil supplementation for optimal cell growth.

It is worth reiterating the UMPS edited population contained unedited or heterozygous cells that are not expected to be auxotrophic, and thus complete lack of growth of UMPS edited cells in uracil deficient media is not expected.

### Example 4. 5-FOA Treatment Selects for UMPS Targeted Cells

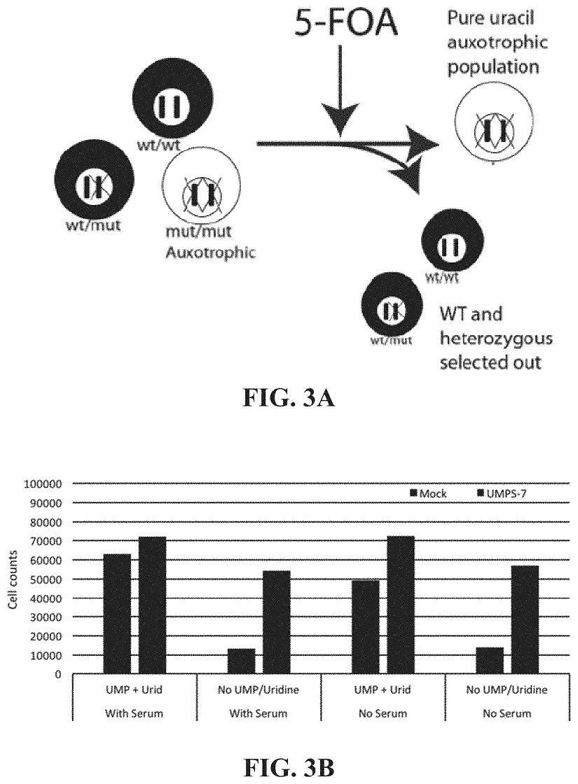

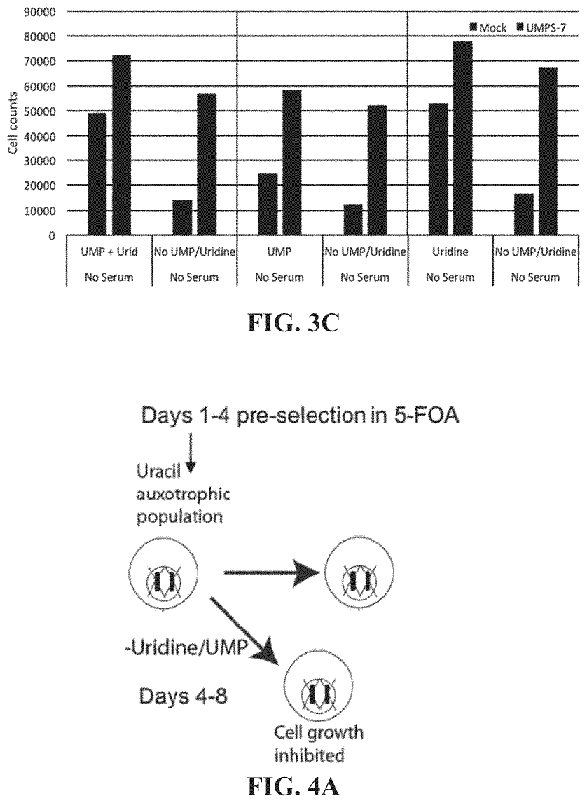

5-FOA selects for uracil auxotrophic cells in other organisms (e.g. Boeke et al. 1984, Mol. Gen. Genet. 197(2):345-6), which is hereby incorporated by reference in its entirety). To investigate the potential utility of 5-FOA for the selection of uracil auxotrophs among human cells, the UMPS gene was targeted in human T cells by Cas9-gRNA complex electroporation followed by recovery (as shown in Example 2) followed by an assay of resistance to 5-FOA treatment (FIG. 3A). Cells were grown in 5-FOA and a variety of combinations of serum and uracil sources for 4 days before cell counting was performed.

Table 10 compares cell counts for cell populations grown with or without serum.

TABLE 10
Cell counts

| | | Average number of | |
|---|---|---|---|
| | | cells per volume unit | |
| Culture condition | Substrates | Mock | UMPS-7 |
| With Serum | UMP + Urid | 63071.71 | 72181.87 |
| With Serum | No UMP/Uridine | 13403.28 | 54282.95 |
| No Serum | UMP + Urid | 49125.44 | 72385.14 |
| No Serum | No UMP/Uridine | 13947.04 | 56895.21 |

Serum, while important for the recovery of cells post electroporation, had no effect on the viability of cells in 5-FOA (FIG. 3B). The cell counts for additional samples grown in 5-FOA without serum are shown in FIG. 3C and Table 11.

TABLE 11
Cell counts

| | Average of cells per volume unit | |
|---|---|---|
| Substrates | Mock | UMPS-7 |
| UMP | 24770.99 | 58299.26 |
| No UMP/Uridine | 12279.07 | 52156.98 |
| Uridine | 53052.43 | 77755.72 |
| No UMP/Uridine | 16467.39 | 67438.73 |

Uridine and UMP improved the survival of both mock treated and UMPS targeted cells in 5-FOA compared to control. This is likely through a competition-based mechanism (uridine can reverse 5-fluorouracil toxicity in humans (see, van Groeningen et al. 1992, Semin. Oncol. 19(2 Suppl 3):148-54, which is hereby incorporated by reference in its entirety)) (FIG. 3B and FIG. 3C). In all cases, UMPS targeted cells exhibited increased survival compared to mock targeted cells. This data indicated that 5-FOA can be used for the selection of uracil auxotrophic cells in a human cell culture.

### Example 5. 5-FOA Selected UMPS Targeted Cells Exhibit Uracil Auxotrophy

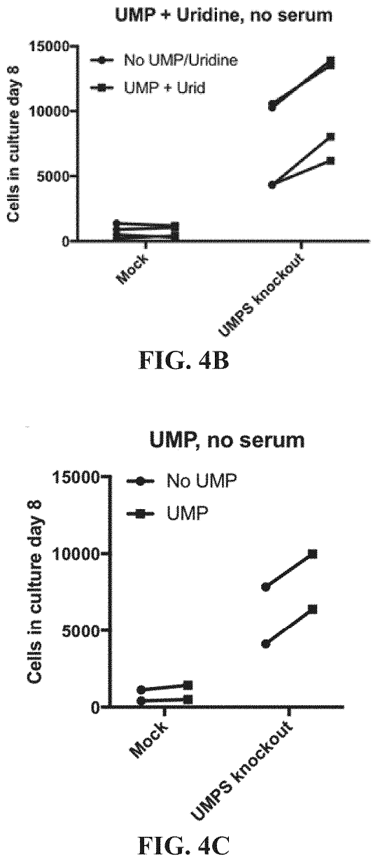

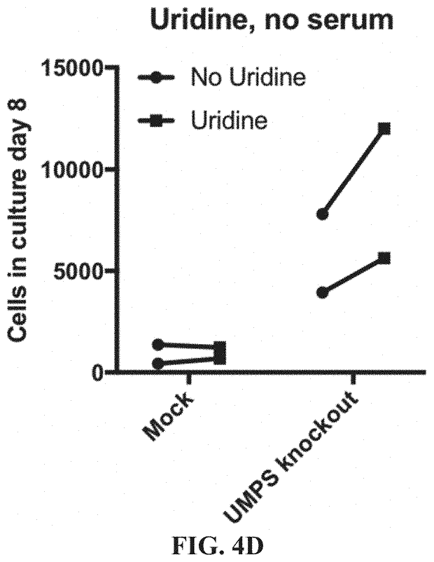

To assay whether or not the cells selected for by 5-FOA treatment were uracil auxotrophs, mock or UMPS targeted T cells were exposed to 5-FOA as shown in Example 4. Following 4 days of 5-FOA selection, the population of cells was split into an uracil containing media (UMP, uridine or both) and an uracil deficient media. A growth assay was subsequently performed by cell counting after following 4 days incubation in test media (Day 8) (FIG. 4A). In all cases, cell growth in the mock targeted cell cultures was negligible and independent of uracil source supplementation—indicating successful killing of non-UMPS targeted cells during the 5-FOA selection step (FIG. 4B-FIG. 4D). In the UMPS targeted population, in all conditions cell growth was stimulated by the addition of uracil and poor cell growth was observed in its absence (FIG. 4B-FIG. 4D).

FIG. 4B compares the cell counts in culture on Day 8 for samples without serum. The values are shown in Table 12.

TABLE 12
Cell counts on Day 8

| | No UMP/Uridine | | | | UMP + Urid | | | |
|---|---|---|---|---|---|---|---|---|
| Replicate | 1 | 2 | 3 | 4 | 1 | 2 | 3 | 4 |
| Mock | 893 | 1365 | 223 | 512 | 1061 | 1185 | 416 | 292 |
| UMPS | 10268 | 10585 | 4318 | 4352 | 13908 | 13526 | 8045 | 6190 |
| knockout | | | | | | | | |

FIG. 4C compares the cell counts in cultures supplemented with UMP and without serum. The values are shown in Table 13.

TABLE 13
Cells counts on Day 8

| | No UMP | | UMP | |
|---|---|---|---|---|
| | Replicate 1 | Replicate 2 | Replicate 1 | Replicate 2 |
| Mock | 1116 | 409 | 1421 | 490 |
| UMPS knockout | 7847 | 4100 | 9978 | 6392 |

FIG. 4D compares the cell counts in cultures supplemented with uridine and without serum. The values are shown in Table 14.

TABLE 14
Cells counts on Day 8

| | No Uridine | | Uridine | |
|---|---|---|---|---|
| | Replicate 1 | Replicate 2 | Replicate 1 | Replicate 2 |
| Mock | 1386 | 431 | 1249 | 687 |
| UMPS knockout | 7795 | 3945 | 12006 | 5629 |

Taken together, the results of Examples 1-5 indicate that editing of the UMPS locus by Cas9 in human T cells generates cells that are dependent on an exogenous uracil source for optimal cell growth. These results demonstrate that engineered human auxotrophy can be used as a mechanism for controlling the proliferation of T cells or some other cell therapy. In addition, 5-FOA selection of UMPS edited cells provides a useful mechanism for selection of a true auxotrophic population of T cells.

### Example 6. Culturing Stem Cells

In order to evaluate another cell type with potential therapeutic relevance, UMPS was engineered in human pluripotent cells. The modified host cells that are the subject matter of the disclosure herein may include stem cells that were maintained and differentiated using the techniques below as shown in U.S. Pat. No. 8,945,862, which is hereby incorporated by reference in its entirety.

Undifferentiated hESCs (H9 line from WICELL®, passages 35 to 45) were grown on an inactivated mouse embryonic fibroblast (MEF) feeder layer (Stem Cells, 2007. 25(2): p. 392-401, which is hereby incorporated by reference in its entirety). Briefly, the cell was maintained at an undifferentiated stage on irradiated low-passage MEF feeder layers on 0.1% gelatin-coated plates. The medium was changed daily. The medium consists of Dulbecco's Modified Eagle Medium (DMEM)/F-12, 20% knockout serum replacement, 0.1 mM nonessential amino acids, 2 mM L-glutamine, 0.1 mM β-mercaptoethanol, and 4 ng/ml rhFGF-2 (R&D Systems Inc., Minneapolis). The undifferentiated hESCs were treated by 1 mg/ml collagenase type IV in DMEM/F12 and scraped mechanically on the day of passage. Prior to differentiation, hESCs were seeded onto MATRIGEL® protein mixture (Corning, Inc.)-coated plates in conditioned medium (CM) prepared from MEF as follows (Nat Biotechnol, 2001. 19(10): p. 971-4, which is hereby incorporated by reference in its entirety). MEF cells were harvested and irradiated with 50 Gy and were cultured with hES medium without basic fibroblast growth factor (bFGF). CM was collected daily and supplemented with an additional 4 ng/ml of bFGF before feeding hES cells.

### Example 7. In Vitro Differentiation of Human Embryonic Stem Cell (ESC)-Endothelial Cells (ECs)

To induce hESC differentiation, undifferentiated hESCs were cultured in differentiation medium containing Iscove's Modified Dulbecco's Medium (IMDM) and 15% defined fetal bovine serum (FBS) (Hyclone, Logan, Utah), 0.1 mM nonessential amino acids, 2 mM L-glutamine, 450 μM monothioglycerol (Sigma, St. Louis, Mo.), 50 U/ml penicillin, and 50 μg/ml streptomycin, either in ultra-low attachment plates for the formation of suspended embryoid bodies (EBs) as previously described (see, Proc Natl Acad Sci USA, 2002. 99(7): p. 4391-6 and Stem Cells, 2007. 25(2): p. 392-401; each of which is hereby incorporated by reference in its entirety). Briefly, hESCs cultured on MATRIGEL® protein mixture (Corning, Inc.) coated plate with conditioned media were treated by 2 mg/ml dispase (Invitrogen, Carlsbad, Calif.) for 15 minutes at 37° C. to loosen the colonies. The colonies were then scraped off and transferred into ultra-low-attachment plates (Corning Incorporated, Corning, N.Y.) for embryoid body formation.

### Example 8. Selection of Auxotrophic Modified Host Cells

The UMPS locus was disrupted in the hESCs by electroporation of Cas9 RNP and selection of a clone with InDels in exon 1 as evaluated by amplification and Sanger sequencing of the genomic locus. For gene editing, hESCs were treated with 10 μm ROCK inhibitor (Y-27632) for 24 hours before electroporation. Cells at 70-80% confluence were harvested with ACCUTASE® solution (Life Technologies). 500,000 cells were used per reaction with a SpCas9 concentration of 150 μg/mL (Integrated DNA Technologies) and a Cas9:sgRNA molar ratio of 1:3 and electroporation performed in P3 Primary Cell solution (Lonza) in 16-well NUCLEOCUVETTE™ Strips in the 4D NUCLEOFECTOR system (Lonza). Immediately after electroporation, cells were transferred into one well of a MATRIGEL® protein mixture (Corning, Inc.)-coated 24 well plate containing 500 μl of mTeSR™ media (STEMCELL Technologies) with 10 μM Y-27632. Media was changed 24 hours after editing and Y-27632 was removed 48 hours after.

Sanger sequencing compared the hESC population before editing, the bulk population after RNP electroporation, and the genotype of the selected clone. Results showed a deletion of 10 bp around the sgRNA target region. The lack of a sequence trace in this region indicated both alleles had been modified.

An auxotrophy assay was performed over four days with different concentrations of uridine. Microscope photos of wells were taken on day 4 after seeding UMPS^{KO/KO} hESCs at similar densities and culturing in the presence of different uridine concentrations. The photos showed that cells proliferated in the presence of 2.5-250 μg/ml but showed no proliferation without added uridine. Quantification of viable cells on day 4 after seeding to evaluate the effect of different uridine concentrations is shown in Table 15.

TABLE 15
Viable cell counts

| Uridine | | Replicate 1 | Replicate 2 | Replicate 3 |
|---|---|---|---|---|
| None | | 0 | 0 | 0 |
| 2.5 | μg/ml | 31040 | 38065 | 45189 |
| 25 | μg/m1 | 31810 | 39635 | 36283 |
| 250 | μg/ml | 19147 | 31050 | 33955 |

Kill curves with different concentrations of supplement versus control were generated to demonstrate that an exogenously supplied version of the product of the knocked-out gene rescues the auxotrophic phenotype of the cell line.

To assess resistance to 5-FOA, the UMPS-KO hESCs were genetically engineered to express GFP from an expression cassette integrated into a safe-harbor locus for easier identification in co-culture with UMPS-WT cells.

A clone that showed bright and stable expression of GFP was selected. These UMPS^{KO/KO} hESCs were mixed with UMPS^{WT/WT} cells that were not expressing GFP and followed up by fluorescence-activated cell sorting (FACS) analysis in the presence of different concentrations of 5-FOA. Table 16 provides counts of viable GFP+ and GFP− cells after culture with different 5-FOA concentrations.

TABLE 16
Viable cell counts

| | | GFP+ | GFP− |
|---|---|---|---|
| None | | 133875 | 121125 |
| 0.25 | μg/ml | 142820 | 5180 |
| 2.5 | μg/ml | 11812.5 | 687.5 |
| 25 | μg/ml | 8455.98 | 334.02 |

Similar to the previous cell types, enrichment for GFP+ cells over time was observed. 54.8% of the cells were GFP+ in the group without 5-FOA, and 95.0% of the cells were GFP+ in the groups with 5-FOA. In this cell type, UMPS-WT cells were sensitive to all tested 5-FOA concentrations, and UMPS-KO cells tolerated the concentration of 0.25 μg/ml well, while showing impaired proliferation at higher concentrations as shown in Table 16.

In conclusion, these results confirm that a key pathway of metabolism may be engineered efficiently to create auxotrophy in a range of human cells from leukemia cell lines to pluripotent cell lines and primary immune cells. Gene targeting of both UMPS alleles may be used to create and purify a cell population with homozygous knockout or enrich those cells using 5-FOA. Cell lines with multiple knockouts and mutations may be also generated to provide rapid multiplexed genome engineering and selection (e.g. 5 auxotrophic mutations and 5 antibiotics).

### Example 9. In Vivo Analysis

In vitro validated auxotrophic knockout cell lines also may be analyzed in vivo. These cell lines are constrained by toxicity and bioavailability of the auxotrophic factor in humans. The gene knockout cell lines are engineered from human T cells or any other lymphocyte. Conditional in vitro growth by the cell line is demonstrated in the presence of the auxotrophic factor, and not in the absence of the auxotrophic factor. The modified mammalian host cells confirmed to be auxotrophic for the factor and capable of expressing the transgene may be administered in a mouse model. Only mice consuming the auxotrophic factor supplement sustain growth of human lymphocytes. Further, cell growth stops in vivo upon removal of nutrient from the mouse food source.

### Example 10. Creating Auxotrophy in Human Cells Through Genetic Engineering

Bioinformatics tools (crispor.tefor.net) were used to identify possible sgRNA target sites in exon 1 of the UMPS gene for spCas9. Putative off-target (OT) effects were predicted using COSMID (crispr.bme.gatech.edu/) (See, Majzner et al. Cancer Cell. 31, 476-485 (2017), which is hereby incorporated by reference in its entirety). Potential off-target sites in the human genome (hg38) were identified using the web-based bioinformatics program COSMID (crispr.bme.gatech.edu) with up to 3 mismatches or 1 bp deletion/insertion with 1 mismatch allowed in the 19 PAM proximal bases. The sgRNAs were ranked by number of highly-similar off-target sites (COSMID score <1) and then ranked by number of OT sites with higher scores. Primers for amplifying all sites were also designed by the COSMID program. All sites were amplified by locus specific PCR, barcoded via a second round of PCR, pooled at equimolar amounts and sequenced using an Illumina MiSeq using 250 bp paired end reads as previously described in Porteus, M. Mol. Ther. 19, 439-441 (2011), which is hereby incorporated by reference in its entirety. The resulting data was analyzed using the custom script indelQuantificationFromFastqPaired-1.0.1.pl(10)(https://github.com/piyuranjan/NucleaseIndelActivityScript/blob/master/indelQuantificationFromFastqPaired-1.0.1.pl).

The 3 sgRNAs with the lowest number of OT sites were identified and used for an in vitro screening of activity. These sgRNAs are shown in Table 17.

TABLE 17
sgRNAs with fewest OT sites

| | Target | SEQ | COSMID | MIT |
|---|---|---|---|---|
| | sequence + | ID | total | Specificity |
| Name | PAM | NO. | OT sites | Score |
| UMPS-3 | CCCCGCAGATCG | 4 | 1 | 96 |
| | ATGTAGAT GGG | | | |
| UMPS-7 | GCCCCGCAGATC | 5 | 6 | 94 |
| | GATGTAGA TGG | | | |
| UMPS-6 | GGCGGTCGCTCG | 6 | 3 | 94 |
| | TGCAGCTT TGG | | | |

sgRNAs were acquired with chemical modifications from Synthego Corporation. The sgRNAs were complexed with Cas9 protein (IDT) at a molar ration of 2.5:1 (sgRNA:protein) and electroporated into activated T cells using a 4D-NUCLEOFECTOR™ system (Lonza). 4 days later, cells were harvested, and genomic DNA extracted using QUICKEXTRACT™ DNA Extraction Kit (Epicentre) according to the manufacturer's protocol. The sgRNA target site was amplified with specific primers (Table 18) and the amplicon sequenced by Sanger sequencing (MCLab, South San Francisco).

TABLE 18
Primers

| Name | Sequence | SEQ ID NO. |
|---|---|---|
| UMPS TIDE | CCCGGGGAAAC | 2 |
| Fwd | CCACGGGTGC | |
| UMPS TIDE | AGGGTCGGTCTG | 3 |
| Rev | CCTGCTTGGCT | |

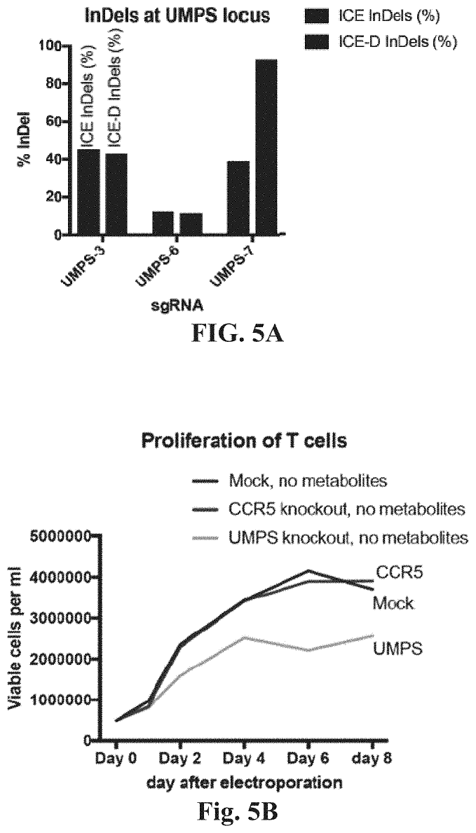

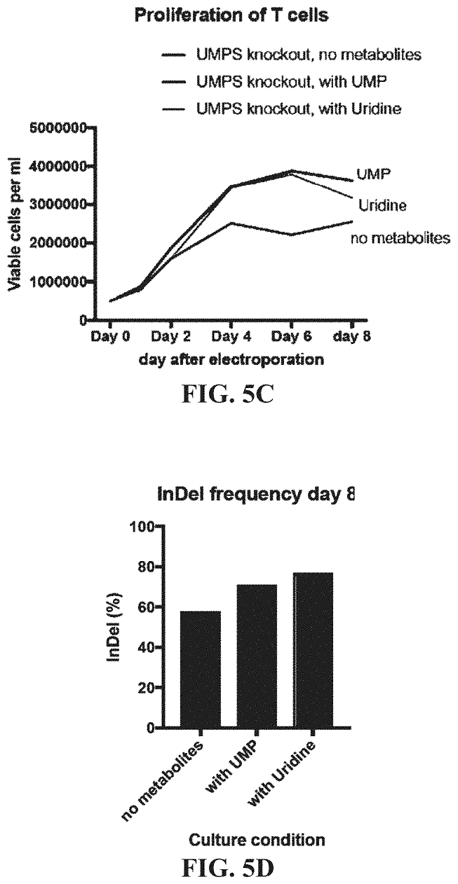

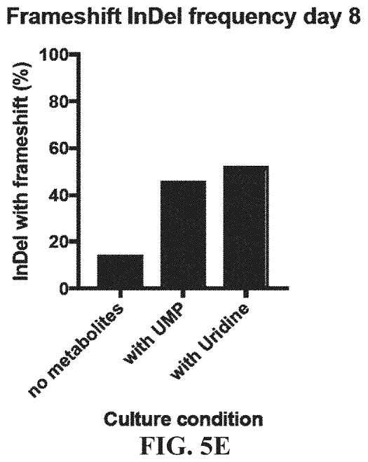

InDel quantification was performed on the sequences using the interference of CRISPR edits (ICE) and ICE-D online tools (ice.synthego.com) (FIG. 5A). Results are shown in Table 19.

TABLE 19
InDel quantification

| | ICE InDels (%) | ICE-D InDels (%) |
|---|---|---|
| UMPS-3 | 45 | 43 |
| UMPS-6 | 12 | 11 |
| UMPS-7 | 39 | 93 |

sgRNA “UMPS-7” was chosen for further experiments. This sgRNA led to the creation of a high proportion of large (greater than 30 bp) deletions that were detectable by inference of CRISPR edits-discordance (ICE-D) but not by conventional ICE or TIDE analysis (www.deskgen.com/landing/tide.html).

To evaluate whether the UMPS knockout leads to differential cell proliferation if cultured without the addition of Uridine or Uridine monophosphate (UMP), the cell counts in culture were followed over time by automatic cell counting with Trypan blue staining. UMPS knockout led to lower cell counts from day 2 after electroporation, compared to cells that were mock electroporated or electroporated using Cas9 targeting a different genomic locus (i.e., CCR5) (FIG. 5B). The cell counts are shown in Table 20.

TABLE 20
InDel quantification

| | Mock, | CCR5 knockout, | UMPS knockout, |
|---|---|---|---|
| Days | no metabolites | no metabolites | no metabolites |
| 0 | 500000 | 500000 | 500000 |
| 1 | 967000 | 835000 | 808000 |
| 2 | 2350000 | 2290000 | 1590000 |
| 4 | 3440000 | 3420000 | 2510000 |
| 6 | 4150000 | 3900000 | 2210000 |
| 8 | 3710000 | 3910000 | 2550000 |

In contrast, cell proliferation was not impaired after UMP S knockout if UMP or Uridine were supplemented at high concentrations (250 μg/ml each) (FIG. 5C). The number of viable cells per ml is shown in Table 21.

TABLE 21
Number of viable cells per ml

| | UMPS knockout, | UMPS knockout, | UMPS knockout, |
|---|---|---|---|
| Day | no metabolites | with UMP | with Uridine |
| 0 | 500000 | 500000 | 500000 |
| 1 | 808000 | 874000 | 769000 |
| 2 | 1590000 | 1860000 | 1590000 |
| 4 | 2510000 | 3470000 | 3430000 |
| 6 | 2210000 | 3880000 | 3790000 |
| 8 | 2550000 | 3630000 | 3170000 |

To confirm the results on the genomic level, genomic DNA was harvested at the end of the experiment and InDels were quantified (FIG. 5D-FIG. 5E). FIG. 5D compares the frequency of InDels in different culture conditions for cells not exposed to 5-FOA. Percentages are shown in Table 22.

TABLE 22
Percentages of overall InDel frequency

| Culture condition | Percent (%) |
|---|---|
| no metabolites | 57.9 |
| with UMP | 71.1 |
| with Uridine | 77.0 |

FIG. 5E compares the frequency of frameshift InDels in different culture conditions for cells not exposed to 5-FOA. Percentages are shown in Table 23.

TABLE 23
Percentages of frameshift InDel frequency

| Culture condition | Percent |
|---|---|
| no metabolites | 14.3 |
| with UMP | 46.1 |
| with Uridine | 52.5 |

Overall InDel frequency was slightly reduced after culture without Uridine or UMP, but when quantifying InDels that would lead to a frameshift (not multiples of +3/−3), there was a reduction of InDels in the cell population without the metabolite addition. This confirms that cells with UMPS knockout due to a frameshift InDel in exon 1 have a disadvantage in survival and proliferation compared to UMPS wild-type cells or cells with InDel in exon 1 that preserved the reading frame.

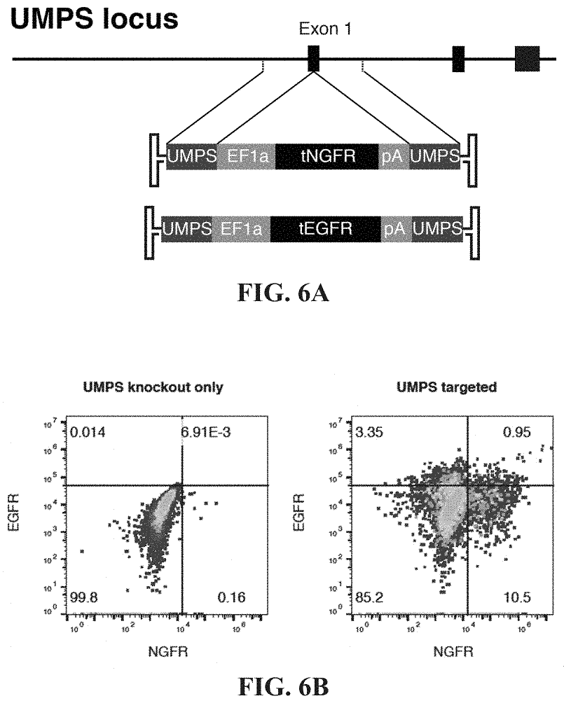

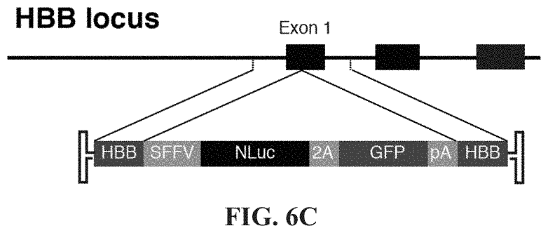

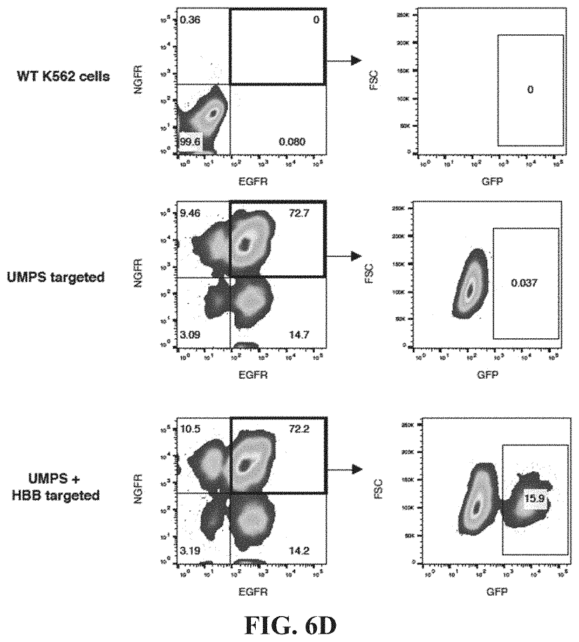

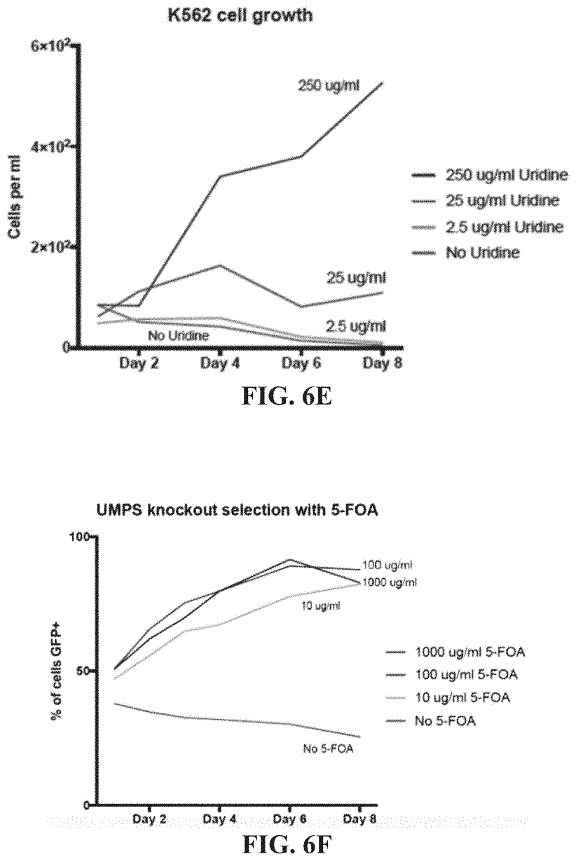

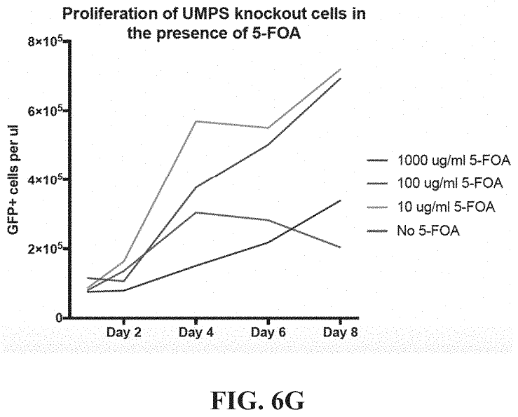

Next, gene targeting constructs were generated that allow the integration of 2 different markers into the UMPS locus, thereby disrupting gene expression and enabling the identification of the cells with bi-allelic gene knockout through co-expression of tEGFR and tNGFR (FIG. 6A), using the approach described in Bak et al., Elife 28:6 (2017), which is hereby incorporated by reference in its entirety. The constructs were cloned by Gibson assembly using standard molecular biology methods with a plasmid backbone that is flanked by the AAV2 inverted terminal repeats (ITRs).

For targeting of stem cells and primary human cells, the constructs were packaged in recombinant adeno-associated virus type 6 (rAAV6) to deliver the DNA after creation of the double-strand break, thereby stimulating homologous recombination to integrate the transgenes. Transfer plasmids for the production of rAAV6 were created by cloning the transgene and surrounding arms homologous to the targeted genomic region into the backbone of pAAV-MCS plasmid (Agilent Technologies) adjacent to the flanking inverted terminal repeats (ITR) by Gibson assembly (NEBUILDER® HiFi DNA Assembly Master Mix, New England Biolabs Inc.). The homology arms were amplified by PCR from healthy donor genomic DNA. For the expression of surface markers, we used the tNGFR and tEGFR (See, Teixeira et al. Curr. Opin. Biotechnol. 55, 87-94 (2019); Chen et al. Sci. Transl. Med. 3 (2011); each of which is hereby incorporated by reference in its entirety). For transcription termination, the poly-adenylation sequence from bovine growth hormone (bGH) was used.

Production of AAV was performed in HEK293T cells by co-transfection of the transfer plasmid with the pdgm6 packaging plasmid and purified by Iodixanol gradient centrifugation. The HEK293 cells were co-transfected with polyethyleneimine with the pDGM6 helper plasmid and the respective transfer plasmid carrying the transgene between homology arms flanked by the AAV2 ITRs. After 48 hours the cells were detached, separated from the supernatant and lysed. The suspension was treated with Benzonase (Sigma Aldrich) and debris pelleted. The crude AAV extract was purified on an Iodixanol density gradient and then subjected to 2 cycles of dialysis against PBS and one cycle against PBS with 5% sorbitol in 1×10⁴ molecular weight cut off (MWCO) SLIDE-A-LYZER™ G2 Dialysis Cassettes (Thermo Fisher Scientific). The AAV titer was determined by extraction of genomic DNA by QUICKEXTRACT™ DNA Extraction Kit (Epicentre) and measuring the absolute concentration of ITR copy numbers by droplet digital PCR (Bio-rad) according to the manufacturers protocol using previously reported primer and probe sets (See, Jaen et al., Mol. Ther. Methods Clin. Dev. 6, 1-7 (2017, which is hereby incorporated by reference in its entirety.).

Targeting with these donor constructs used as plasmids was first tested in the myeloid leukemia cell line K562 (ATCC® CCL-243™). The cells were electroporated with 2 μg of each plasmid on a SF Cell Line 4D NUCLEOFECTOR™ system (Lonza) following the manufacturer's protocol. When targeting the 2 markers into the UMPS locus, a small but stable population of cells that showed co-expression of both markers was identified (FIG. 6B).

Magnetic bead enrichment was used to sequentially enrich for the cells expressing the surface markers EGFR and NGFR. For magnetic separation, cells expressing both tNGFR and tEGFR were enriched by sequential magnetic bead sorting using antibodies against NGFR and EGFR with PE and APC as fluorochromes (Biolegend), the Anti-phycoerythrin (PE MultiSort kit (Miltenyi) and anti-APC MicroBeads (Miltenyi) on LS or MS columns (Miltenyi). FACS sorting was performed on an FACS ARIA™ II SORP cell sorter (BD Biosciences).

To make identification easier, a second editing step was performed in which an expression cassette with firefly luciferase and TurboGFP was targeted into a safe harbor locus (HBB) (FIG. 6C). The K562 cells were suspended in 20 ul SF cell line solution with 6 μg Cas9 protein (IDT) and 3.2 μg sgRNA (Trilink) and electroporated. After resuspension in K562 cell medium (RPMI with 10% BGS and supplemented with GLUTAMAX™ and Penicillin/Streptomycin), the cells were transduced with rAAV carrying the expression cassette. This resulted in a cell population expressing all 3 markers (tNGFR, tEGFR and GFP) that were sorted by flow cytometry. Results of the flow cytometry are shown in FIG. 6D. The percent of GFP+ cells in each group is shown in FIG. 6F.

The sorted UMPS^{KO/KO}/GFP⁺ cell population were subjected to assays evaluating their auxotrophy and their resistance to 5-FOA. The cells were split into samples of equal numbers and cultured in the presence of different concentrations of Uridine or without. With supplementation of high concentrations of Uridine (250 μg/ml) the cells expanded rapidly. Cell growth was inhibited at a lower concentration (25 μg/ml) while cell numbers declined with a lower concentration or no Uridine (FIG. 6E). The number of cells per ml is shown in Table 24.

TABLE 24
Number of cells per ml from Day 1 to Day 8

| | 250 ug/ml Uridine | | | 25 ug/ml Uridine | | | 2.5 ug/ml Uridine | | | No Uridine | | |
|---|---|---|---|---|---|---|---|---|---|---|---|---|
| Day 1 | 83.41 | 109.11 | 64.28 | 60.92 | 69.81 | 58.10 | 57.83 | 49.52 | 40.56 | 131.21 | 103.97 | 18.04 |
| Day 2 | 130.60 | 80.43 | 39.92 | 150.58 | 73.78 | N/A | 99.62 | 40.41 | 31.70 | 97.77 | 28.14 | 29.30 |
| Day 4 | 520.75 | 356.31 | 142.97 | 305.15 | 114.37 | 71.23 | 124.37 | 33.19 | 20.39 | 89.31 | 24.21 | 13.69 |
| Day 6 | 474.67 | 460.12 | 205.32 | 146.01 | 56.14 | 43.75 | 46.59 | 10.77 | 8.05 | 35.06 | 3.98 | 4.99 |
| Day 8 | 631.12 | 629.35 | 318.17 | 242.61 | 46.45 | 39.19 | 28.68 | 2.06 | 1.85 | 15.82 | 0.54 | 0.48 |

The same experiment was performed with Nalm6 cells and a similar dependency on the uridine concentration in the culture was observed that was not visible for cells with intact UMPS. Table 25 provides data of growth curves of UMPS^{KO/KO} Nalm6 cells cultured with different uridine concentrations. No difference was observed between the groups receiving uridine supplement treatment and those not for wild-type cells.

TABLE 25
Cell counts of UMPS^{KO/KO} Nalm6 cells

| | Day | | | | | | | |
|---|---|---|---|---|---|---|---|---|
| | 0 | 1 | 2 | 3 | 4 | 5 | 6 | 7 |
| UMPS-WT, 250 μg/ml uridine | 17980 | 33784 | 59524 | 167715 | 303894 | 1163678 | 3511660 | 7293447 |
| | 24390 | 40000 | 58737 | 160160 | 213547 | 1115507 | 3058542 | N/A |
| UMPS-WT, 25 μg/ml uridine | 19505 | 39683 | 70175 | 194185 | 311891 | 1173622 | 2708995 | 5746352 |
| | 36496 | 40000 | 68027 | 174292 | 376249 | 1216000 | 3792593 | 7585185 |
| | 27548 | 31847 | 70922 | 214190 | 447240 | 1413856 | 3792593 | 6320988 |
| UMPS-WT, 2.5 ug/ml uridine | 20052 | 35088 | 67340 | 156019 | 244368 | 923391 | 2017336 | 3869992 |
| | 19661 | 34130 | 38314 | 153257 | 225750 | 1119256 | 3269476 | 6117085 |
| | 21142 | 28986 | 62598 | 137812 | 338624 | 1148582 | 3269476 | 6538953 |
| UMPS-WT, no Uridine | 13496 | 15343 | 43860 | 86496 | 252167 | 1048734 | 3058542 | 6772487 |
| | 14582 | 23529 | 31974 | 93077 | 278867 | 1138455 | 3511660 | 7585185 |
| | 14140 | 18994 | 45045 | 111810 | 316049 | 1223111 | 3511660 | 6538953 |
| 250 μg/ml uridine | 20704 | 37175 | 101781 | 210805 | 551249 | 2072099 | 3792593 | 7585185 |
| | 24510 | 35461 | 106667 | 265340 | 551249 | 2343280 | 4514991 | 6320988 |
| | 29762 | 30769 | 112045 | 257649 | 564374 | 1844675 | 4122383 | 7901235 |
| 25 μg/ml uridine | 14984 | 21277 | 69085 | 142944 | 388585 | 1624178 | 3646724 | 7293447 |
| | 18952 | 25189 | 68027 | 176018 | 324708 | 1360329 | 3386243 | 5746352 |
| | 14400 | 19688 | 71301 | 189125 | 395062 | 1360329 | 3792593 | 6538953 |
| 2.5 μg/ml uridine | 20121 | 42194 | 69808 | 70287 | 80625 | 193482 | 380782 | 755497 |
| | 29155 | 38023 | 88300 | 70547 | 81737 | 188952 | 383866 | 796763 |
| | 28090 | 40650 | 87146 | 78663 | 79012 | 193913 | 377748 | 793429 |
| No uridine | 10258 | 13661 | 13222 | 11234 | 14667 | 24413 | 27139 | 40429 |
| | 12284 | 16244 | 13126 | 11518 | 15131 | 21683 | 27762 | 46573 |
| | 12427 | 15772 | 15585 | 11729 | 16441 | 25738 | 29410 | 43139 |

Significantly greater growth was observed in the groups supplemented with uridine, especially the groups supplemented with 25 μg/ml and 250 μg/ml uridine.

To determine the resistance of UMP S knockout cells to 5-FOA the purified uMPS^{KO/KO}/GFP⁺ K562 cells were mixed at an equal ratio with UMPS^{wT/WT}/GFP-negative K562 cells. The cells were cultured in the presence of Uridine and different concentrations of 5-FOA (FIG. 6F). Table 26 provides the percentages of GFP-positive (+) cells under different culture conditions.

TABLE 26
Percentages of GFP+ cells

| | 1000 ug/ml | | 100 ug/ml | | 10 ug/ml | | No | |
|---|---|---|---|---|---|---|---|---|
| | 5-FOA | | 5-FOA | | 5-FOA | | 5-FOA | |
| Day 1 | 51.9 | 49.4 | 52.6 | 48.9 | 45.4 | 48.5 | 38.1 | 37.6 |
| Day 2 | 65.0 | 58.9 | 67.1 | 64.2 | 54.7 | 56.8 | 34.6 | 34.9 |
| Day 3 | 72.9 | 66.9 | 79.5 | 71.4 | 65.6 | 64.0 | 33.6 | 31.7 |
| Day 4 | 82.0 | 77.7 | 85.2 | 74.4 | 64.8 | 69.7 | 32.4 | 31.5 |
| Day 6 | 92.6 | 90.5 | 92.1 | 86.2 | 79.1 | 76.5 | 31.2 | 29.2 |
| Day 8 | 90.1 | 75.7 | 92.8 | 82.8 | 85.0 | 79.8 | 24.8 | 26.0 |

FIG. 6G shows the growth curve for GFP+ cells at different amounts of 5-FOA. The values are show in Table 27.

TABLE 27
Number of GFP + cells per μl

| | 1000 ug/ml | 100 ug/ml | 10 ug/ml | |
|---|---|---|---|---|
| | 5-FOA | 5-FOA | 5-FOA | No 5-FOA |
| Day 1 | 75925.72 | 115272.35 | 87013.04 | 80080.81 |
| Day 2 | 79080.77 | 106377.73 | 163245.74 | 135616.84 |
| Day 4 | 151010.55 | 376993.53 | 569281.05 | 304640.45 |
| Day 6 | 217794.10 | 501940.62 | 550780.31 | 282520.65 |
| Day 8 | 339282.35 | 693093.53 | 719624.13 | 203799.66 |

At all concentrations that were used, the fraction of UMPS^{KO/KO} cells increased over time. Cells with UMP knockouts proliferated well at the concentrations 10 and 100 μg/ml of 5-FOA, while the highest concentration slowed their cell growth down.

### Example 11. UMPS Editing Creates Auxotrophy in T Cells and Allows for Selection with 5-FOA

T cells were isolated from buffy coats that were acquired from the Stanford Blood Center (Palo Alto, Calif.) using Ficoll density gradients and MACS negative selection (Miltenyi T cell enrichment kit). The T cells were cultured in X-VIVO15 medium supplemented with 5% human serum (Sigma) and 100 IU/ml IL-2.

Before electroporation, T cells were activated for 3 days with Anti-CD3/-CD28 beads (STEMCELL Technologies), also referred to as Dynabeads in the art, and IL-2 (100 IU/ml). Activation beads were removed by magnetic immobilization before electroporation. K562 cells and Nalm6 cells were kept in logarithmic growth phase before electroporation. sgRNAs were acquired from Synthego with 2′-O-methyl-3′-phosphorothioate modifications at the three terminal nucleotides of both ends (See, Bonifant, et al. Mol. Ther.—Oncolytics. 3, 16011 (2016), which is hereby incorporated by reference in its entirety).

The two selection markers, tEGFR and tNGFR, were targeted into the UMPS locus in primary human T cells after isolation of CD3+ T cells from healthy donors and activation of the cells.

Large-scale sgRNAs were acquired high-performance liquid chromatography (HPLC)-purified. High-fidelity (HiFi) Cas9 protein was purchased from IDT. The sgRNAs were complexed with HiFi spCas9 protein (IDT) at a molar ratio of 2.5:1 (sgRNA:protein) and electroporated into the cell lines or activated T cells using a 4D-NUCLEOFECTOR™ System (Lonza) in 16-cuvette strips.

For targeting of transgenes into specific loci of the genome, cells were edited as described, resuspended directly after electroporation in 80 μl of medium, then incubated with rAAV6 for transduction at multiplicities of infection (MOI) of 5000 vg/cell. After 8-12 hours, the suspension was diluted with medium to reach a cell concentration of 0.5-1E6 cells per ml. For targeting of the HBB locus, a previously characterized sgRNA with the target sequence CTTGCCCCACAGGGCAGTAA (SEQ ID NO: 7) was used (See, Teixeira et al., Curr. Opin. Biotechnol. 55, 87-94 (2019), which is hereby incorporated by reference in its entirety).Cas9 and sgRNA were complexed to an RNP and mixed with the T cells resuspended in P3 buffer and electroporated in the 4D NUCLEOFECTOR™ system (Lonza) using program EO-115. Human T cells are known in the art to allow high editing frequencies at low toxicity as described in Bak et al., 2018, to create a population of cells with a bi-allelic UMPS knockout using RNP/rAAV6 gene targeting methods. Cells expressing both markers were simultaneously expressed. The following cell counts per electroporation, electroporation solutions and programs were used: 2E5 K562 cells in SF cell line solution using program FF-120, 2E5 Nalm6 cells in SF-cell line solution and program CV-104 and 1E6 activated T cells in P3 solution. For controls edited at the CCR5 locus the genomic target sequence of the sgRNA was GCAGCATAGTGAGCCCAGAA (SEQ ID NO: 8). After electroporation, the cells were resuspended in medium and rAAV added.

Three days after targeting, a population of EGFR+/NGFR+ cells was identified and expanded by co-culturing with Anti-CD3/-CD28 magnetic beads in the presence of high Uridine concentrations. The population of EGFR+/NGFR+ cells was differentiated from cells that received AAV alone due to brighter expression indicating stable integration as opposed to episomal expression from AAV.

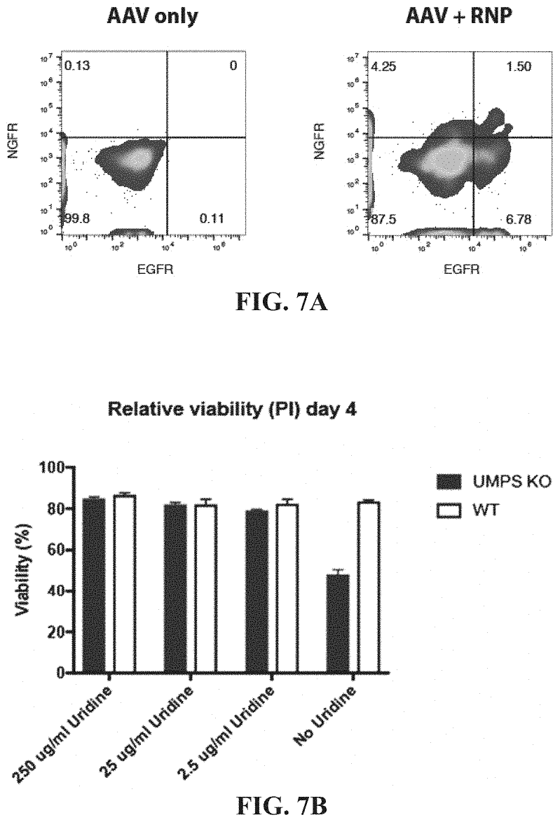

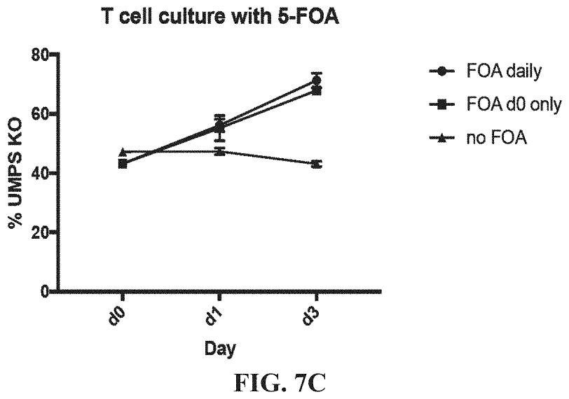

After expansion, the EGFR+/NGFR+ population was sorted using flow cytometry to get a population of T cells with bi-allelic UMPS knockout. Results are shown in FIG. 7A.

These T cells were also subjected to an auxotrophy assay and the possibility to select these cells with 5-FOA was tested. When culturing the cells in the presence of Anti-CD3/-CD28 beads and different concentrations of Uridine, cells proliferated only in the presence of Uridine, which confirmed their auxotrophic cell growth. Higher Uridine concentrations led to higher proliferation rates. Auxotrophic growth of UMPS KO or wild-type (WT) T cells is shown in in FIG. 7B and Table 28.

TABLE 28
Viable cells per ml

| | UMPS KO | | | WT | | |
|---|---|---|---|---|---|---|
| 250 μg/ml | 319120 | 345862 | 348022 | 609575 | 412493 | 468354 |
| uridine | | | | | | |
| 25 μg/ml | 282368 | 268684 | 304864 | 384503 | 410116 | 338547 |
| uridine | | | | | | |
| 2.5 μg/ml | 226596 | 217594 | 224448 | 486192 | 362626 | 364194 |
| uridine | | | | | | |
| No uridine | 46037 | 45351 | 52771 | 424742 | 414301 | 393938 |

Table 29 show the relative viability of the cell population on Day 4.

TABLE 29
Viable cells per ml

| | UMPS KO | | | WT | | |
|---|---|---|---|---|---|---|
| 250 μg/ml | 85.66 | 85.29 | 84.38 | 86.05 | 86.97 | 87.62 |
| Uridine | | | | | | |
| 25 μg/ml | 82.16 | 81.57 | 83.09 | 83.91 | 83.40 | 79.66 |
| Uridine | | | | | | |
| 2.5 μg/ml | 78.78 | 79.28 | 79.74 | 84.64 | 82.19 | 80.45 |
| Uridine | | | | | | |
| No Uridine | 46.06 | 48.32 | 50.46 | 83.78 | 84.15 | 82.96 |

To evaluate the possibility to select for UMPS^{KO/KO} cells with 5-FOA, the sorted cells were mixed with wild-type cells, which were labeled with different tracking dyes, and cultured in the presence or absence of 5-FOA. In part of the samples, 5-FOA was only added on the first day (Day 0) while in another group it was supplemented daily. Table 30 and FIG. 7C show the percent (%) of the UMPS-KO T cells (labelled with eFluor670) over time when culturing with or without 5-FOA.

TABLE 30
Percent of UMPS^{KO/KO} cells in mixed cell population

| | 5-FOA daily | | | 5-FOA Day 0 only | | | no 5-FOA | | | | | |
|---|---|---|---|---|---|---|---|---|---|---|---|---|
| Day 0 | 43.2 | | | 43.2 | | | 47.2 | | | | | |
| Day 1 | 58.5 | 55.4 | 54.5 | 59.2 | 55.6 | 50.8 | 46.5 | 48.0 | 49.3 | 46.5 | 47.5 | 46.4 |
| Day 3 | 74.0 | 70.7 | 69.2 | 67.2 | 68.6 | 67.8 | 43.4 | 42.9 | 43.1 | 43.8 | 44.2 | 41.7 |

Groups were compared for statistically significant differences using an unpaired t test. No statistical significance was observed between the groups treated with 5-FOA, while there was a significant increase in the percent of UMPS-KO T cells in the treated groups compared to the untreated group.

In both 5-FOA treated groups, the fraction of cells with UMPS knockout increased over time, indicating their increased resistance to the compound compared to wild-type cells, and that a one-time treatment with 5-FOA was sufficient to lead to an enrichment of modified cells over several days. The data in Table 30 illustrates 5-FOA selects for T cells with UMPS knockout.

In fact, FACS analysis of a culture of a mixed population of UMPS knockout and wild-type T cells with 5-FOA. UMPS-KO T cells were labeled with eFluor670, and wild-type cells were labeled with carboxyfluorescein succinimidyl ester (CFSE). Results showed that on Day 0 in the group treated with 5-FOA only on the first day (Day 0), 43.7% of the cells were UMPS-KO T cells, while 56.0% were observed to the wild-type cells. On Day 0 in the control group not treated with 5-FOA, 47.7% of the cells were UMPS-KO T cells, and 52.1% were wild-type cells. On Day 3 in the group with 5-FOA supplemented daily, 74.0% of the cells were UMPS-KO T cells, while 25.8% were wild-type cells. On Day 3 in the control group not treated with 5-FOA, 43.1% of the cells were UMPS-KO T cells, and 56.4% were wild-type cells.

### Example 12. Cellular Therapy

Pluripotent stem cells are genetically engineered to make them dependent on externally supplied factors. These cells are injected into immunodeficient NSG mice as teratoma-forming assays to evaluate the safety system, which prevents teratoma formation through withdrawal of the externally supplied compound. Cell lines used are iPSCs: iLiF3, iSB7-M3 (source: Nakauchi Lab at Stanford University), and hES: H9.

### Example 13. Teratoma-Forming Assay in Gastrocnemius Muscle

To determine whether the safety switch can eradicate teratomas that originate from pluripotent cells, iPSCs or ES cells that were genetically modified (or control cells) were transplanted into mice. The cells expressed luciferase for in vivo detection. 1×10⁶ UMPS-engineered hESCs were resuspended in a 100 μl of MATRIGEL® protein mixture (Corning, Inc.) and PBS mixture and injected into the gastrocnemius muscle of the right hind leg of anesthetized NSG mice. The mice were followed up for tumor formation by tumor size measurement and by bioluminescence imaging. After establishment of tumors, whether withdrawal of Uridine triacetate (UTA) led to tumor regression was tested. At the endpoint (tumor sizes above 1.7 cm or impairment of mouse activity, otherwise 24 weeks) tumor was explanted and fixated for histological analysis.

### Example 14. K562 Xenograft Model

For the K562 xenograft assay, 6 to 12 weeks old male NOD SCID gamma mouse (NSG) mice were transplanted with 1×10⁶ K562 cells resuspended in MATRIGEL® protein mixture (Corning, Inc.) 1:1 diluted with PBS under anesthesia. All animals were kept and handled according to institutional guidelines and the experimental protocol was approved by Stanford University's Administrative Panel on Laboratory Animal Care.

The growth of UMPS^{KO/KO} engineered cells was analyzed in vivo after transplantation into a model organism by supplying the animal with high doses of uridine. Uridine has been used in humans for the treatment of hereditary orotic aciduria and for toxicity from fluoropyrimidine overdoses (see, van Groeningen, et al. Ann. Oncol. 4, 317-320 (1993); Becroft, et al. J. Pediatr. 75, 885-91 (1969); each of which are hereby incorporated by reference in its entirety), but it is poorly absorbed in the gastrointestinal tract and broken down in the liver (See, Gasser, et al. Science. 213, 777-8 (1981); each of which are hereby incorporated by reference in its entirety). Its bioavailability can be increased by administration as the prodrug uridine triacetate (UTA, PN401), which has FDA approval for the above-mentioned indications (See, Weinberg et al., PLoS One. 6, e14709 (2011); Ison et al., Clin. Cancer Res. 22, 4545-9 (2016); each of which is hereby incorporated by reference in its entirety). In humans and mouse models, this can effectively increase uridine serum levels by greater than 10-fold (See, Garcia et al., Brain Res. 1066, 164-171 (2005); FDA, “XURIDEN—Highlights of prescribing information.” (2015), (available at https://www.accessdata.fda.gov/drugsatfda_docs/label/2015/208169s0001b1.pdf); each of which is hereby incorporated by reference in its entirety.

The previously engineered UMPS^{KO/KO} K562 cell line expressing firefly luciferase (FLuc) was used in a xenograft model in NSG mice. Control K562 cells with wild-type UMPS were engineered by targeting an expression cassette with FLuc and GFP into a safe-harbor locus, in order to establish comparable xenograft models for both UMPS genotypes in which the tumors can be monitored by bioluminescence imaging. Cas9 RNP is targeted to exon 1 of the HBB locus with a guide RNA and a DNA donor template transduced by rAAV6 which carries a FLuc-2A-GFP-polyA cassette under control of the SFFV promoter. FACS analysis was performed four days after targeting of K562 cells to evaluate GFP expression before sorting of the GFP+ population. In a control group administered the AAV only, 1.61% of the cells were GFP+, and 13.4% of the cells.

Mice were fed with either regular mouse food or with a custom food which had been enriched with 8% (w/w) UTA, an amount that had previously been shown to increase serum levels in mice while being well tolerated (See, Garcia et al., 2005). UTA was acquired from Accela ChemBio Inc. and added to make the 8% (w/w) to Teklad mouse food (Envigo) and the food irradiated before use. Control food was the standard mouse food Teklad 2018 (irradiated).

Alternatively, the food was supplemented with uridine monophosphate. These cells may be implanted into the immunocompromised mice in a local (hind leg) or systemically through an intravenous (iv) injection.

UMPS^{KO/KO} K562 cells or control cells were transplanted subcutaneously and observed weekly with bioluminescence imaging. Luminescence imaging of K562 cells was performed 5 minutes after intraperitoneal (ip) injection of 125 mg/kg D-Luciferin (PerkinElmer) on an IVIS Spectrum imaging system (PerkinElmer). The localized growth that has been described for K562 cells after subcutaneous xeno-transplantation was observed (See, Sontakke, et al. Stem Cells Int. 2016, U.S. Pat. No. 1,625,015 (2016), which is hereby incorporated by reference in its entirety). Mice were euthanized when they got moribund or if longest tumor diameter exceeded 1.75 cm. Except for one mouse with engraftment failure, an increase in tumor burden in UMPS wild-type cells with both normal or UTA supplemented food was observed. In contrast, luminescence of UMPS^{KO/KO} K562-derived tumors were observed to only increased in the mice fed with 8% UTA, while tumor burdens were observed to remain stable in the majority of mice that received food without UTA.

Auxotrophic cell proliferation of the UMPS^{KO/KO} engineered hES cells in vivo was also analyzed. Except for one mouse with failure to form a teratoma, masses were observed in all the mice fed with supplemented UTA, after injection of the pluripotent cells into the hind legs of NSG mice. When euthanizing the mice 7 weeks after cell injection, large teratomas that had formed in the region of injection in mice fed with UTA were extracted, while in mice on normal food the teratomas were visible but significantly smaller and weighed less as shown in Table 31. Bone marrow is analyzed at the time that the animal dies or is sacrificed (latest 16 weeks after injection). Table 31 shows quantification results of teratoma weights (p<0.05 by unpaired t-test comparing all mice between groups, p<0.01 when censoring the mouse without engraftment). Groups were compared by statistical tests as indicated using Prism 7 (GraphPad).

TABLE 31
Teratoma weight

| | | | | | |
|---|---|---|---|---|---|
| Mouse No. | 1 | 2 | 3 | 4 | 5 |

Weight [g]

| | | | | | |
|---|---|---|---|---|---|
| No UTA | 603 | 311 | 468 | 91 | 174 |
| With UTA | 3108 | 33 | 2923 | 1545 | 937 |

The in vivo results were consistent with the previous in vitro results, which had shown reduced but not completely abrogated proliferation of UMPS^{KO/KO} cells at the uridine concentration of 2.5 μg/ml (=10 nmol/ml). This concentration corresponds to serum uridine levels of mice, which are reported in the literature to range from 8 to 11.8 nmol/ml (See, Karle, et al. Anal. Biochem. 109, 41-46 (1980), which is hereby incorporated by reference in its entirety).

Overall, these results are evidence a metabolic auxotrophy can be engineered to add a control mechanism over cell proliferation of human cells both in vitro and in vivo.

### Example 15. GvHD Model

Whether the safety system can prevent the side effects of xeno-GvHD is determined in a mouse model. Genetically modified human T cells or control T cells are transplanted into irradiated immunocompromised mice and mice are supplied with UTA or not. Mice are evaluated for weight loss or other signs of GvHD and sacrificed upon establishment of disease (latest 16 weeks). Cells are followed by bioluminescence imaging and blood draws.

### Example 16. Enzyme Replacement Therapy in Lysosomal Storage Disease (LSD)

Pluripotent stem cells are genetically engineered to encode for an enzyme of interest integrated at UMPS locus to make them dependent on externally supplied uridine. Individuals in need of enzyme replacement therapy for the specific enzyme to treat a LSD are administered compositions comprising these cells along with uridine, to promote expression of the enzyme that is deficient in the individual. The dosing and timing of the administration of uridine is adjusted based on the desired expression of the enzyme.

In some instances, cells are genetically engineered to encode for an enzyme of interest at HLCs locus to make them dependent on externally supplied biotin.

While embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the disclosure described herein may be employed in practicing the subject matter of the disclosure. It is intended that the following claims define the scope of the disclosure herein and that methods and structures within the scope of these claims and their equivalents be covered thereby.

What is claimed is:

1. A donor template comprising:
(a) one or more nucleotide sequences homologous to a fragment of an auxotrophy-inducing locus, or homologous to the complement of said auxotrophy-inducing locus, and
(b) a transgene encoding a protein or a nucleic acid.

2. The donor template of claim 1, wherein the donor template is single stranded.

3. The donor template of claim 1, wherein the donor template is double stranded.

4. The donor template of claim 1, wherein the donor template is comprised within a plasmid or a viral vector.

5. The donor template of claim 4, wherein the viral vector is a retroviral, lentiviral, adenoviral, adeno-associated viral, or herpes simplex viral vector.

6. The donor template of claim 1, wherein the transgene is flanked on both sides by the one or more nucleotide sequences homologous to a fragment of the auxotrophy-inducing locus or the complement thereof.

7. The donor template of claim 1, wherein the auxotrophy-inducing locus is a gene encoding a protein that is involved in synthesis, recycling or salvage of an auxotrophic factor.

8. The donor template of claim 1, wherein the auxotrophy-inducing locus is within a gene in Table 1 or within a region that controls expression of a gene in Table 1.

9. The donor template of claim 1, further comprising an expression control sequence operably linked to the transgene encoding the protein or nucleic acid.

10. The donor template of claim 9, wherein the expression control sequence is a tissue-specific expression control sequence.

11. The donor template of claim 9, wherein the expression control sequence is a promoter or enhancer.

12. The donor template of claim 9, wherein the expression control sequence is an inducible promoter.

13. The donor template of claim 9, wherein the expression control sequence is a constitutive promoter.

14. The donor template of claim 9, wherein the expression control sequence is a posttranscriptional regulatory sequence.

15. The donor template of claim 9, wherein the expression control sequence is a microRNA.

16. The donor template of claim 1, wherein the transgene encodes a marker gene.

17. The donor template of claim 16, wherein the marker gene comprises at least a fragment of NGFR or EGFR, at least a fragment of CD20 or CD19, Myc, HA, FLAG, GFP, or an antibiotic resistance gene.

18. The donor template of claim 1, wherein the transgene encodes a therapeutic protein or a therapeutic nucleic acid.

19. The donor template of claim 1, wherein the transgene encodes a T cell antigen receptor.

20. The donor template of claim 1, wherein the transgene encodes an antisense RNA, siRNA, aptamer, microRNA mimic, anti-miR, or synthetic mRNA.

21. A modified host cell ex vivo, comprising: a transgene encoding a protein or a nucleic acid integrated at an auxotrophy-inducing locus, wherein said modified host cell is auxotrophic for an auxotrophic factor and capable of expressing the protein or a nucleic acid.

22. The modified host cell of claim 21 that is a human cell.

23. The modified host cell of claim 21, wherein the modified host cell is selected from the group consisting of: an embryonic stem cell, a stem cell, a progenitor cell, a pluripotent stem cell, an induced pluripotent stem (iPS) cell, a somatic stem cell, a differentiated cell, a mesenchymal stem cell, a neural stem cell, a hematopoietic stem cell or a hematopoietic progenitor cell, an adipose stem cell, a keratinocyte, a skeletal stem cell, a muscle stem cell, a fibroblast, a NK cell, a B-cell, a T cell, and a peripheral blood mononuclear cell (PBMC).

24. The modified host cell of claim 21 that is derived from cells from a subject to be treated with the modified host cells.

25. A method of producing a modified mammalian host cell comprising: (a) introducing into said mammalian host cell (i) a first nuclease system that targets and cleaves DNA at an auxotrophy-inducing locus, or (ii) a first nucleic acid encoding the first nuclease system, and (b) a donor template of claim 1.

26. The method of claim 25, further comprising introducing (i) a second nuclease system that targets and cleaves DNA at a second genomic locus, or (ii) a second nucleic acid encoding said second nuclease system.

27. The method of claim 25, wherein the nuclease system comprises a ZFN.

28. The method of claim 25, wherein the nuclease system comprises a TALEN.

29. A method of producing a modified mammalian host cell comprising: introducing into a mammalian host cell: (a) a Cas9 polypeptide, or a nucleic acid encoding said Cas9 polypeptide, (b) a guide RNA specific to an auxotrophy-inducing locus, or a nucleic acid encoding said guide RNA, and (c) a donor template of claim 1.

30. The method of claim 29, further comprising introducing into said mammalian host cell: (a) a second guide RNA specific to a second auxotrophy-inducing locus, or a nucleic acid encoding said second guide RNA, and optionally (b) a second donor template.

31. The method of claim 29, wherein the guide RNA is a chimeric RNA.

32. The method of claim 29, wherein the guide RNA comprises two hybridized RNAs.

33. The method of claim 25, further comprising expanding a population of the mammalian host cells before or after step (a) and (b).

34. The method of claim 25, further comprising (c) selecting cells that contain the transgene integrated into the auxotrophy-inducing locus.

35. The method of claim 34, wherein the selecting comprises one or both of: (i) selecting for cells that require the auxotrophic factor to survive; and (ii) selecting for cells that comprise the transgene integrated into the auxotrophy-inducing locus.

36. The method of claim 34, wherein the auxotrophy-inducing locus is a gene encoding uridine monophosphate synthetase and the selecting comprises selecting against cells lacking the transgene integrated into the auxotrophy-inducing locus by contacting the cells with 5-FOA.

37. A composition comprising one or more of a donor template targeting an auxotrophy-inducing locus, a nuclease system that targets and cleaves DNA at the auxotrophy-inducing locus, or a nucleic acid encoding the nuclease system, and sterile water or a pharmaceutically acceptable excipient.

38. A composition comprising: a modified mammalian host cell comprising a transgene integrated into an auxotrophy-inducing locus and sterile water or a pharmaceutically acceptable excipient.

39. A method of expressing a therapeutic factor in a subject comprising:
(a) administering to the subject modified mammalian host cells comprising a transgene integrated into an auxotrophy-inducing locus, the transgene comprising or encoding the therapeutic factor; and
(b) administering an auxotrophic factor corresponding to the auxotrophy-inducing locus to the subject.

40. The method of claim 39, wherein administering the modified mammalian host cells and auxotrophic factor is performed concurrently.

41. The method of claim 39, wherein administering the modified mammalian host cells and auxotrophic factor is performed sequentially.

42. The method of claim 39, further comprising continuing administration of said auxotrophic factor regularly for a period of time sufficient to promote expression of the therapeutic factor.

43. The method of claim 39, further comprising decreasing administration of said auxotrophic factor to decrease expression of the therapeutic factor.

44. The method of claim 39, further comprising increasing administration of said auxotrophic factor to increase expression of the therapeutic factor.

45. The method of claim 39, further comprising discontinuing administration of said auxotrophic factor to create conditions that result in growth inhibition or death of the modified mammalian host cells.

46. The method of claim 39, further comprising temporarily interrupting administration of said auxotrophic factor to create conditions that result in temporary growth inhibition of the modified mammalian host cells.

47. The method of claim 39, further comprising continuing administering of said auxotrophic factor for a period of time sufficient to exert a therapeutic effect in a subject.

48. The method of claim 39, wherein the modified mammalian host cells are regenerative.

49. The method of claim 39, wherein the administration of the modified mammalian host cells comprises localized delivery.

50. The method of claim 39, wherein the administration of the auxotrophic factor comprises systemic delivery.

51. The method of claim 39, further comprising deriving the mammalian host cells from the subject prior to modification.

52. A method of treating a subject with a disease, a disorder, or a condition comprising: administering to the subject (a) modified mammalian host cells comprising a transgene integrated into an auxotrophy-inducing locus, the transgene comprising or encoding a therapeutic factor, and (b) an auxotrophic factor corresponding to the auxotrophy-inducing locus in an amount sufficient to produce expression of a therapeutic amount of the therapeutic factor.

53. The method of claim 52, wherein the disease, the disorder, or the condition is selected from the group consisting of: cancer, Parkinson's disease, graft versus host disease (GvHD), autoimmune conditions, hyperproliferative disorder or condition, malignant transformation, liver conditions, genetic conditions including inherited genetic defects, juvenile onset diabetes mellitus, and ocular compartment conditions.

54. The method of claim 48, wherein the disease, the disorder, or the condition affects at least one system of the body selected from the group consisting of muscular, skeletal, circulatory, nervous, lymphatic, respiratory, endocrine, digestive, excretory, and reproductive systems.

55. The method of claim 52, wherein the auxotrophy-inducing locus is within a gene encoding uridine monophosphate synthetase (UMPS).

56. The method of claim 52, wherein the auxotrophic factor is uridine.

57. The method of claim 52, wherein the auxotrophy-inducing locus is within a gene encoding holocarboxylase synthetase (HLCS).

58. The method of claim 52, wherein the auxotrophic factor is biotin.

59. The method of claim 52, wherein the modified mammalian host cells are embryonic stem cells, stem cells, progenitor cells, pluripotent stem cells, induced pluripotent stem (iPS) cells, somatic stem cells, differentiated cells, mesenchymal stem cells, neural stem cells, hematopoietic stem cells or hematopoietic progenitor cells, adipose stem cells, keratinocytes, skeletal stem cells, muscle stem cells, fibroblasts, NK cells, B-cells, T cells or peripheral blood mononuclear cells (PBMCs).

60. The method of claim 52, wherein the modified mammalian host cells are derived from the subject to be treated with the modified host cells.

61. The method of claim 52, wherein administering the modified mammalian host cells and the auxotrophic factor occurs concurrently.

62. The method of claim 52, wherein the modified mammalian host cells and the auxotrophic factor are administered sequentially.

63. The method of claim 52, wherein administration of the auxotrophic factor is continued regularly for a period of time sufficient to promote therapeutic expression of the therapeutic factor.

64. The method of claim 52, wherein administration of the auxotrophic factor is decreased to decrease expression of the therapeutic factor.

65. The method of claim 52, wherein administration of the auxotrophic factor is increased to increase expression of the therapeutic factor.

66. The method of claim 52, further comprising discontinuing administration of the auxotrophic factor to induce growth inhibition or cell death of the modified mammalian host cells.

67. The method of claim 52, wherein administration of the auxotrophic factor is continued for a period of time sufficient to exert a therapeutic effect in the subject.

68. The method of claim 52, wherein the modified mammalian host cells are regenerative.

69. The method of claim 52, wherein the administration of the modified mammalian host cells or the auxotrophic factor comprises localized delivery.

70. The method of claim 52, wherein the administration of the auxotrophic factor comprises systemic delivery.

71. The method of claim 52, wherein the disease is a lysosomal storage disease (LSD).

72. The method of claim 71, wherein the lysosomal storage disease (LSD) is Gaucher's Disease (Type 1/2/3), MPS2 (Hunter's) disease, Pompe disease, Fabry disease, Krabbe disease, Hypophosphatasia, Niemann-Pick disease type A/B, MPS1, MPS3A, MPS3B, MPS3C, MPS3, MPS4, MPS6, MPS7, Phenylketonuria, MLD, Sandhoff disease, Tay-Sachs disease, or Battens disease.

73. The method of claim 52, wherein the therapeutic factor is Glucocerebrosidase, Idursulfase, Alglucosidase alfa, Agalsidase alfa, Agalsidase beta, Galactosylceramidase, Asfotase alfa, Acid Sphingomyelinase, Laronidase, heparan N-sulfatase, alpha-N-acetylglucosaminidase, heparan-α-glucosaminide N-acetyltransferase, acetylglucosamine 6-sulfatase, Elosulfase alfa, Glasulfate, B-Glucoronidase, Phenylalanine hydroxylase, Arylsulphatase A, Hexosaminidase-B, Hexosaminidase-A, or tripeptidyl peptidase 1.

74. The method of claim 52, wherein the disease is Friedreich's ataxia, Hereditary angioedema, or Spinal muscular atrophy.

75. The method of claim 52, wherein the therapeutic factor is frataxin, C1 esterase inhibitor or SMN1.

76. A method of reducing the size of a tumor or reducing a rate of growth of a tumor in a subject, the method comprising: administering to the subject a modified mammalian host cell comprising a transgene integrated into an auxotrophy-inducing locus, the transgene comprising or encoding a therapeutic factor, and an auxotrophic factor corresponding to the auxotrophy-inducing locus in an amount sufficient to produce expression of a therapeutic amount of the therapeutic factor.

77. The method of claim 29, further comprising expanding a population of the mammalian host cells before or after step (a) and (b).

78. The method of claim 77, further comprising (c) selecting cells that contain the transgene integrated into the auxotrophy-inducing locus.

79. The method of claim 78, wherein the selecting comprises one or both of: (i) selecting for cells that require the auxotrophic factor to survive; and (ii) selecting against cells lacking the transgene integrated into the auxotrophy-inducing locus.

80. The method of claim 79, wherein the auxotrophy-inducing locus is a gene encoding uridine monophosphate synthetase and the selecting against cells lacking the transgene integrated into the auxotrophy-inducing locus comprises contacting the cells with 5-FOA.


SEQUENCE LISTING (8 sequences)

SEQ ID NO: 1 - RNA, 100 nt, Artificial Sequence
  source: /note=“Description of Artificial Sequence Synthetic guide RNA”
gccccgcagaucgauguagaguuuuagagcuagaaauagcaaguuaaaauaaggcuaguccguuaucaacuugaaaaaguggcaccgagucggugcuuuu

SEQ ID NO: 2 - DNA, 21 bp, Artificial Sequence
  source: /note=“Description of Artificial Sequence Synthetic UMPS-O-1 sequencing oligonucleotide”
cccggggaaacccacgggtgc

SEQ ID NO: 3 - DNA, 23 bp, Artificial Sequence
  source: /note=“Description of Artificial Sequence Synthetic UMPS-O-2 sequencing oligonucleotide”
agggtcggtctgcctgcttggct

SEQ ID NO: 4 - DNA, 23 bp, Homo sapiens
ccccgcagatcgatgtagatggg

SEQ ID NO: 5 - DNA, 23 bp, Homo sapiens
gccccgcagatcgatgtagatgg

SEQ ID NO: 6 - DNA, 23 bp, Homo sapiens
ggcggtcgctcgtgcagctttgg

SEQ ID NO: 7 - DNA, 20 bp, Homo sapiens
cttgccccacagggcagtaa

SEQ ID NO: 8 - DNA, 20 bp, Homo sapiens
gcagcatagtgagcccagaa